(19) United States
(12) Patent Application Publication
(10) Pub. No.: US 2023/0407296 A1
(43) Pub. Date: Dec. 21, 2023

(54) Method to Regulate ALOX12-AS1 IncRNA-Mediated Alteration of ALOX12 Protein and to Screen for Novel Drugs to Alter ALOX12 Protein Level

(71) Applicant: Mohammad Golam Sabbir, Winnipeg (CA)

(72) Inventor: Mohammad Golam Sabbir, Winnipeg (CA)

(21) Appl. No.: 17/884,202

(22) Filed: May 2, 2023

Related U.S. Application Data
(60) Provisional application No. 63/209,072, filed on Jun. 10, 2021.

(51) Int. Cl. C12N 15/113; C12Q 1/6883

(52) U.S. Cl. CPC C12N 15/113; C12Q 1/6883; C12N 2310/11; C12Q 2600/136; C12Q 2600/158

(57) ABSTRACT

The arachidonate 12-lipoxygenase (ALOX12) enzyme catalyzes polyunsaturated fatty acids and facilitates generation of bioactive lipid mediators associated with various biological processes and disease pathologies. The human genome assembly revealed that the ALOX12 gene overlaps an antisense non-coding gene designated as ALOX12-antisense 1 (ALOX12-AS1). This arrangement indicates that the uncharacterized ALOX12-AS1 long non-coding RNA (lncRNA) may bind to the sense coding ALOX12 mRNA to form an antisense-sense duplex providing the basis of a novel ALOX12 regulatory mechanism. This represents a novel regulatory mechanism for the modulation of potent bioactive lipid mediators that contribute to both health and disease.

### PRIOR APPLICATION INFORMATION

The instant application claims the benefit of U.S. Provisional Patent Application Ser. No. 63/209,072, filed Jun. 10, 2021, and entitled “A method to regulate ALOX12-AS1 lncRNA-mediated alteration of ALOX12 protein and to screen for novel drugs to alter ALOX12 protein level”, the entire contents of which are incorporated herein by reference for all purposes.

### BACKGROUND OF THE INVENTION

Arachidonate 12-lipoxygenase (ALOX12) is a member of the lipoxygenase family of proteins originally discovered in blood platelets [1, 2] and highly expressed in the epidermal cells of human [3, 4] and murine skin [5]. ALOX12, a non-heme iron-containing dioxygenase, catalyzes the stereo-specific addition of oxygen onto polyunsaturated fatty acids, e.g. linoleic, arachidonic, and docosahexaenoic (DHA) acids, generating bioactive lipids that regulate a variety of biological processes [6-9]. Abnormal ALOX12 expression and altered ALOX12-mediated metabolites are linked to multiple human pathologies [10], including cardiovascular disease [6, 11], skin disorders [12], non-alcoholic fatty liver disease [13, 14], diabetic nephropathy [15], cancer [16-19] and Alzheimer's disease [20]. Despite its role in metabolic regulation and association with human diseases, the tissue and cell-type-specific regulation of ALOX is not clearly understood. However, there is limited evidence that the transcription factor p63 induces ALOX12 expression in epidermal cells [21] while the hematopoietic transcription factor RUNX1 attenuates ALOX12 expression in platelets [22].

The ALOX12 gene is located in a cluster with several other arachidonate lipoxygenase genes (ALOXE3, ALOX12B, ALOX15, and ALOX15B) in an ˜7 Mbp region on the short arm (petite) of human chromosome 17 (Chr.17p13.1) [23]. Interestingly, a gene antisense to ALOX12 and designated as ALOX12 antisense-1 (ALOX12-AS1) has been identified within this cluster [24]. Based on the human genome assembly GRCh38/hg38 [25], ALOX12-AS1 generates multiple long-noncoding RNAs (lncRNAs) antisense to the ALOX12 gene [24]. The existence of ALOX12-AS1 lncRNA is supported by cloned cDNAs using poly-A RNAs derived from human embryos (GeneBank ID: AK074580.1 [26] and DA571112.1 [27]), and human cell lines (GeneBank ID: BU596417.1 [27]). However, there has been no report in the literature characterizing the tissue and cell-type-specific expression of ALOX12-AS1 lncRNA, except that, Human protein atlas-based transcriptome analysis listed expression of ALOX12 in a large variety of organs/tissues [28], The functionality of ALOX12-AS1 lncRNA is not known [24]. Antisense lncRNAs that overlap with a protein-coding gene typically interfere with sense transcription through direct polymerase II transcriptional interference in cis [29, 30]. Antisense lncRNA can also post-transcriptionally regulate the sense gene by a variety of mechanisms including regulation of mRNA stability, microRNA accessibility, and recruitment of polysome or translational activators/repressors [31].

### SUMMARY OF THE INVENTION

According to a first aspect of the invention, there is provided a method for treating a disease or disorder associated with Arachidonate 12-lipoxygenase (ALOX12) protein levels, said method comprising:
administering an effective amount of an ALOX12-AS1 modulating compound to an individual in need of such treatment, the effective amount altering ALOX12 mRNA-ALOX12-AS1 lncRNA interaction and thereby altering ALOX12 protein levels compared to an untreated control.

According to another aspect of the invention, there is provided a method for determining if a patient is a candidate for further screening for a disease characterized by abnormal ALOX12 protein levels comprising:
measuring expression levels of ALOX-AS1 lncRNA and a reference lncRNA in a blood sample taken from the patient;
comparing the patient ALOX12-AS1 lncRNA expression level to a control ALOX12-AS1 lncRNA expression level, thereby providing an ALOX12-AS1 lncRNA ratio;
comparing the patient reference lncRNA expression level and to a control reference lncRNA expression level, thereby providing a reference lncRNA ratio, wherein:
if the ALOX12-AS1 lncRNA ratio is greater than the reference lncRNA ratio, the individual is screen for an acute or chronic inflammatory disease;
if the ALOX12-AS1 lncRNA ratio is lower than the reference lncRNA ratio, the individual is screened for a decreased inflammatory response disease.

According to another aspect of the invention, there is provided a method for determining if a compound of interest is an ALOX12 modulating compound comprising:
(a) growing a culture of cells in the presence of a compound of interest;
(b) after a suitable growth interval, measuring ALOX12 protein levels in the cells of the culture;
(c) comparing the ALOX12 protein levels to control ALOX12 protein levels from a culture of similar cells grown under similar growth conditions except for presence of the compound of interest, wherein
(d) if ALOX12 protein levels are elevated compared to the control ALOX12 protein levels, the compound is an ALOX12 activator; or
(e) if ALOX12 protein levels are reduced compared to the control ALOX12 protein levels, the compound is an ALOX12 inhibitor.

The compound identified in step (d) may then be characterized for ALOX12-AS1 lncRNA nucleo-cytoplasm translocation activation.

The compound identified in step (e) may then be characterized for ALOX12-AS1 lncRNA nucleo-cytoplasm translocation inhibition.

### BRIEF DESCRIPTION OF THE DRAWINGS

FIG. 1: ALOX12-AS1 lncRNA expression in human tissues and cell lines. (A): Diagrammatic representation of human chromosome 17 showing location and exon-intron structure of nine ALOX12-AS1 lncRNA isoforms. Four reverse (R) primers, designated as 201R, 202R, 208R, and 209R were combined with a common forward (F) primer designated as 209F, to amplify nine ALOX12-AS1 isoforms. The amplicon length specific to each isoform is mentioned in the Table in F. (B-E): Agarose gel images showing amplification of ALOX12-AS1 isoforms and GAPDH in different human tissues. cDNAs equivalent to 100 ng total RNA were used in each RT-PCR reaction amplified over 35 cycles. The same stock cDNAs were used in all experiments. L(bp): DNA ladder (base pairs). AD: adipose, AR: artery, BM: bone marrow, CE: cerebellum, CO: cortex, IN: intestine, LI: liver, SM: skeletal muscle, and SK: skin tissues respectively. The isoform labelling in B&C were color coded to match the colors of the arrows which indicate individual PCR products potentially associated with specific isoforms. The red arrows in B indicate the bands chosen for Sanger sequencing corresponding to the dominant ALOX12-AS1-201 and -202 isoform-specific amplicons (See Figure S1). (F): Summary of ALOX12-AS1 isoform expression in different human tissues. The relative degree of expression was arbitrarily designated as high (H), moderate (M) and low (L) based on visual estimation of band intensities. Hyphen: not detected (below detection limit). Abbreviations for human tissues are provided in B. (G): Northern blots showing expression of full-length ALOX12-AS1-201/202 lncRNAs in A431, EA.hy926, HEK293, HepG2, and THP-1 cells. The single-strand RNA ladder (denatured) was marked on the blot. (H): RNA-FISH images showing intracellular localization of ALOX12-AS1 lncRNAs in HEK293 cells. Green arrow: micronucleus; cyan arrow: metaphase spread; white arrow: nucleolus.

FIG. 2: RNA-FISH showing intracellular localization of ALOX12-AS1-201/202 lncRNA in EA.hy926 and HepG2 cells. (A-I): High magnification RNA-FISH images showing intracellular localization of ALOX12-AS1 lncRNA in EA.hy926 (A-G) and HepG2 (H&I) cells. The interpretation of colored arrows is as follows. Yellow arrow: cytosol; pink arrow: nuclear speckles, orange arrow: micronucleus, red arrow: metaphase plate (chromosomes); dotted red arrow: early-metaphase; black arrow: late anaphase, white arrow: nucleolus, cyan arrow: metaphase spread, cyan and white dotted arrows: differential level of lncRNA expression in the HepG2 nucleus.

FIG. 3: RNA-FISH showing ALOX12-AS1-201/202 lncRNA expression and localization in THP-1 cells. (A-B): High magnification RNA-FISH images showing expression of ALOX12-AS1-201/202 lncRNA in THP-1 cells. Yellow arrows: cytosol (note that the nucleus in these cells appears very condensed, possibly apoptotic or under a specific physiological state); White arrows: nucleolus; Pink arrows: nuclear speckles; Cyan arrows: dividing cell/metaphase; Green arrows: micronucleus. Note the presence of lncRNA in the micronuclei of THP-1 cells. (C): Table summarizing tissue-specific expression and intracellular localization of ALOX12-AS1 lncRNA isoforms based on panels A and B and Supplementary Figure S8. Asterisk: cytosolic localization mainly in the apoptotic cell, “+”: positive localization. R/UD: reduced/undetected.

FIG. 4: Nucleolar, nucleoplasm and cytosolic localization of ALOX12-AS1 lncRNAs in EA.hy926 cells. (A): Bright field images showing THP-1 cells in isotonic and hypotonic buffers as well as homogenized cells showing isolated nuclei. Nucleus: red arrows, nucleoli: yellow arrows. (B) Immunofluorescence images showing nucleolar structures in HEK293, THP-1, and EA.hy926 cells. Yellow arrow: nucleolus. (C): Immunofluorescence images showing isolated and purified nucleoli. Cyan arrow: collection of isolated enriched nucleoli. (D): Immunoblots showing the relative abundance of NPM1 (nucleolar), Histone H3 (nuclear), α-tubulin (cytosol), and RPS6 (ribosomal) proteins in different subcellular fractions. HMW: high molecular weight fraction of NPM1 exclusively present in the total nuclear fraction and nucleoplasm but undetectable in the nucleolar fraction. (E): Oriole-stained total protein profile showing differential protein fingerprints in the different subcellular fractions. (F-G): Agarose gel images showing amplification of ALOX12-AS1 isoforms in different subcellular fractions derived from EA.hy926 cells. cDNAs equivalent to 100 ng total RNA were used in each RT-PCR reaction amplified over 35 cycles. L(bp): DNA ladder (base pairs). (H): Scatter plot showing the copy number of the lncRNAs in different subcellular compartments in different cell-types. P values by one-way ANOVA followed by Dunnett's multiple comparisons test; ****P≤0.0001.

FIG. 5: TPA-induced differentiation of THP-1 cells to macrophages associated with nucleo-cytoplasmic translocation of ALOX12-AS1 lncRNAs as well as suppression of ALOX12 protein level. (A): Immunoblots showing ALOX12, ITGAM, NPM1 and RPS6 protein levels in THP-1 cells treated with vehicle (DMSO) and 100 nM TPA for 1, 3, 24 and 72 hours. (B): Immunofluorescence images showing CD68 expression and localization in THP-1 cells and TPA-induced MLCs at different time points following drug treatment. Yellow arrows: membrane localization of CD68; cyan arrows: endosomal localization of CD68. (C): FISH images showing translocation of ALOX12-AS1-201 lncRNAs to the cytoplasm in TPA-induced MLCs at different time points following drug treatment. The yellow and cyan arrows indicate nucleolar and cytosolic localization of the lncRNA, respectively. (D): Quantitative RT-PCR showing copy numbers of ALOX12-AS1-201/206 isoforms per 100 ng total RNA derived from the cytosolic and nuclear fractions. N=9, 3 replicates from 3 independent experiments. P values by one-way ANOVA followed by Dunnett's multiple comparisons test. ****P≤0.0001. Ns: p>0.05 (not significant). (E): Immunoblots showing relative abundance of ALOX12 and NPM1 in the vehicle (DMSO)-treated THP-1 cells and TPA-treated (100 nM for 72 hour) MLCs. (F): Scatter plot showing relative abundance of ALOX12. N=9, 3 replicates from 3 independent experiments. P values by t-test (unpaired). ****P≤0.0001.

FIG. 6: ALOX12 activity assay, RNase protection assay, and detection of ALOX12 and ribosomal proteins in ALOX12-AS1-201 pull down products from the cytosol. (A): Immunoblots showing the presence of ALOX12 and FGG in unactivated human platelets. NS: non-specific. The bottom panel represents total protein loading. B: Bar graph showing ALOX12 enzyme activity in 100 μg total protein lysate derived from unactivated human platelets, TPA/vehicle treated (100 nM for 1 hour) THP-1 cells. The ALOX12 activity was measured as 12S-HETE production from 50 μM arachidonic acid substrate in 15 min at 37° C. PLT: platelet. Data represent Mean±SD, N=three replicates. P values by one-way ANOVA followed by Dunnett's multiple comparisons test. ****P≤0.0001, *P=0.03, and ns=not significant. (C): Diagrammatic representation of the ALOX12 and ALOX12-AS1 genomic locus. Red colored rectangle: exon. Scale in base pairs. Blue and red dotted lines represent overlapped sense and antisense exons of the respective genes and their corresponding sequences (SEQ ID NO: 1, 2). Arrow: 5′-3′ polarity of the polynucleotide strands. (D) Agarose gel (1%) showing individual mRNAs and ALOX12+ALOX12-AS1 duplex treated with RNaseA/T1. Kbp: Kilobase pairs. (E): Diagrammatic representations of the ALOX12-AS1-201 pull down strategy. (F): Oriole-stained gel showing ALOX12-AS1 lncRNA pull down proteins in HEK293 cells. (G): Immunoblot showing presence of RPS6 in the ALOX12-AS1 lncRNA pull down product in HEK293 cells. (H): Agarose gel showing multiplex RT-PCR products indicating presence of ALOX12 mRNA in the ALOX12-AS1 lncRNA pull down product in HEK293 cells.

FIG. 7: Knockdown of ALOS12-AS1 isoforms and its effect on ALOX12 expression in HEK293 cells. (A): Table showing ALOX12-AS1-201/202 isoform-specific ASOs. Each ASO contains a central core of phosphorothioate-modified DNA (highlighted black) flanked by 2′-O-methyl (2′OMe) modified RNA bases (highlighted in Red). *: phosphorothioate linkage; m: 2′OMe modification. (B-C): The predicted secondary structures of single-stranded ALOX12-AS1-201 and -202 lncRNAs. The RNA secondary structure was generated using RNAfold Webserver provided by the Institute of Theoretical Chemistry, University of Vienna, Austria [32]. The 5′ and 3′ end of each lncRNA isomer is mentioned in the base pair number. The relative positions of the ASOs are shown by red lines. The blue highlights indicate the region of each isoform which was amplified by PCR in the multiple RT-PCR. (D-F and L-N): Agarose gel showing multiple-RT PCR generated products. (G&Q): Immunoblots showing ALOX12 protein levels. Bottom panels represent protein loading in corresponding immunoblots. A red arrow in Q indicates the bands used for the relative quantification of ALOX12. (H-J, and O): Scatter plots showing copy numbers of ALOX12-AS1 lncRNA isoforms and ALOX12 mRNA, respectively. Data represents mean±SEM, n=10, 3-4 replicates from 3 independent experiments. CTRL: untransfected. P-values in G-I are obtained by t-test (unpaired). The p-value in O was obtained by one-way ANOVA test with post-hoc multiple comparisons. (K&P): Scatter plots showing relative ALOX12 protein levels. Data represents mean±SEM, n=10, 3-4 replicates from 3 independent experiments. CTRL: untransfected. P-values in G-I were obtained by t-test (unpaired).

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

The arachidonate 12-lipoxygenase (ALOX12) enzyme catalyzes polyunsaturated fatty acids and facilitates generation of bioactive lipid mediators associated with various biological processes and disease pathologies. The human genome assembly revealed that the ALOX12 gene overlaps an antisense non-coding gene designated as ALOX12-antisense 1 (ALOX12-AS1). This arrangement indicates that the uncharacterized ALOX12-AS1 long non-coding RNA (lncRNA) may bind to the sense coding ALOX12 mRNA to form an antisense-sense duplex providing the basis of a novel ALOX12 regulatory mechanism. Therefore, this study was designed to determine whether the interaction of ALOX12-AS1 with ALOX12 mRNA functions as an anti-sense/sense duplex-mediated regulatory mechanism controlling the cellular content of ALOX12. Our findings indicate that two major isoforms of ALOX12-AS1 lncRNA are ubiquitously expressed in a variety of primary adult human tissues and different transformed cell types. RNA-FISH revealed cell-type-specific cytosolic as well as nuclear and nucleolar localization of the lncRNA. Interestingly, Phorbol ester-induced nucleo-cytoplasmic translocation of the lncRNA in monocytic THP-1 cells resulted in a reduction of ALOX12 protein without a concomitant change in its mRNA level. This indicated ALOX12-AS1 operates via an antisense-sense duplex-mediated translational downregulation mechanism. This deduction was validated by demonstrating sense/antisense duplex formation and an association of the duplex with ribosomal proteins in HEK293 cells. Overall, this study revealed a hitherto unknown mechanism of antisense lncRNA-mediated translational downregulation of ALOX12 that adds to the existing regulatory mechanisms for the modulation of potent bioactive lipid mediators that contribute to both health and disease.

As discussed above, the findings reported in this study indicate a previously uncharacterized ALOX12-AS1 lncRNA that is ubiquitously expressed in human tissues and differentially distributed within the cell depending on cell-type and isoform-specificity. A functional role for this antisense lncRNA in translational regulation of the overlapping ALOX12 gene provides a mechanism by which the generation of potent lipid mediators by ALOX12 might be regulated, thus making this process biologically relevant within a broader context. It is also important to realize that apart from this regulatory role of the antisense lncRNA, the enzymatic activity of ALOX12 and subsequent production of bioactive lipids can be constrained by the availability of the precursor polyunsaturated fatty acids and downstream effectors such as glutathione peroxidase, which reduces 12S-HpETE to 12S-HETE [9].

This is the first study to report that ALOX12 protein level can be decreased by an overlapping antisense lncRNA ALOX12-AS1 through translational downregulation which leads to functional consequence in terms of reduced lipoxygenase enzymatic activity. Furthermore, this mechanism involves nucleo-cytoplasmic translocation of ALOX12-AS1 in response to the physiological stimulus (phorbol ester) that controls PKC signaling pathways. The tissue-specific expression and cell-type-specific intracellular localization of this lncRNA indicates that ALOX12-AS1 may have a diverse and ubiquitous functional role beyond controlling ALOX12. In this context, the abundance of ALOX12-AS1 lncRNA in different intracellular compartments, including cytosol, nucleolus, and nuclear speckles, indicates subcellular localization may specify function. Our findings also suggest that it may be possible to therapeutically target ALOX12-AS1 to manipulate ALOX12 protein level in various metabolic tissues, such as adipose and liver, as well as blood vessels. The clinical implication is evident in light of our recent finding that bioactive lipids generated by ALOX12 influence cardiovascular disease risk in relation to sex and obesity [33]. Overall, the findings of this study open up a hitherto unknown avenue of research involving antisense lncRNA-mediated regulation of an important lipoxygenase.

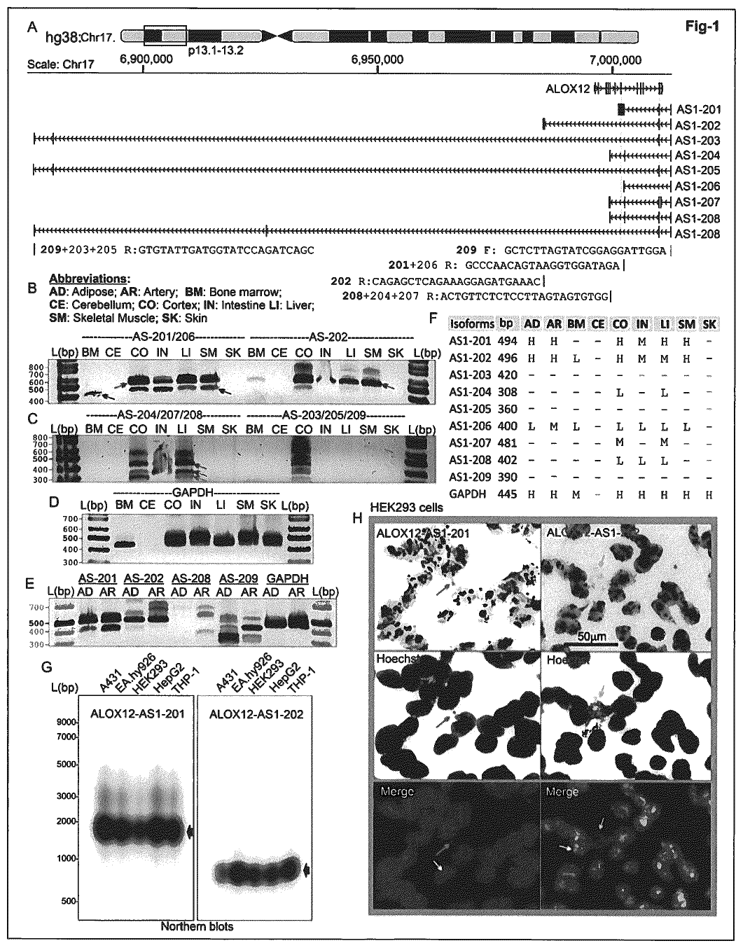

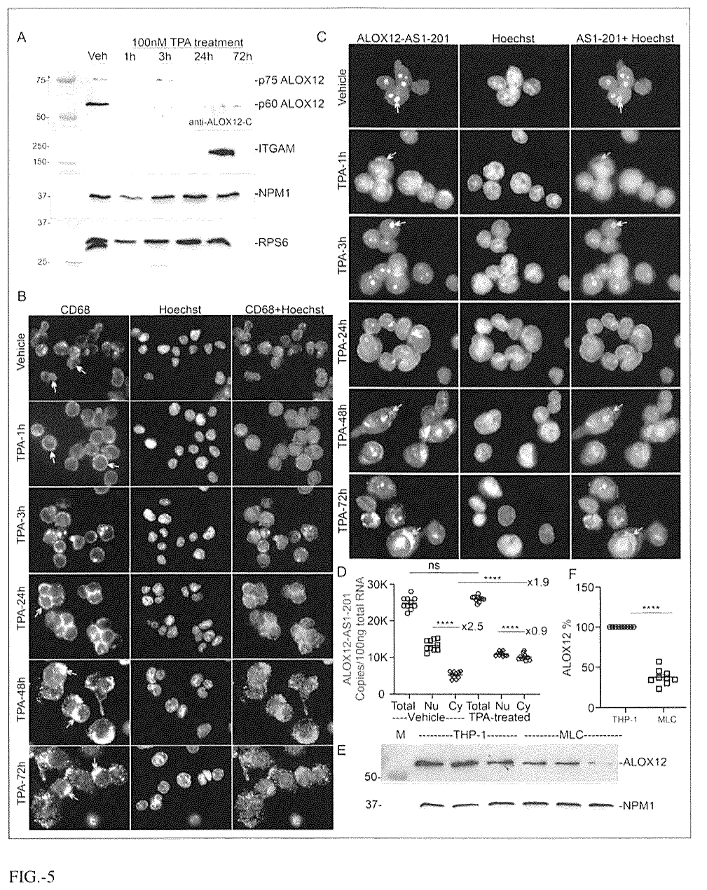

ALOXs are a heterogeneous class of stereo-specific lipid peroxidizing enzymes [24]. There are six functional ALOX genes (ALOXE3, ALOX5, ALOX12, ALOX12B, ALOX15, and ALOX15B) and all, except ALOX5, are clustered on chromosome 17p13.1-13.2 within a 3.5 Mbp (4.6 Mbp-8.2 Mbp) region [34]. Most of the ALOX genes are successively arranged and do not overlap each other, however, ALOX12-AS1 and the protein-coding ALOX12 gene are overlapped (FIG. 1A), which is suggestive of a regulatory function. Antisense lncRNA-mediated regulation of overlapping sense protein coding gene can operate at the transcriptional or post-transcriptional levels. Transcriptional interference occurs through direct polymerase II transcriptional interference in cis [29, 30], whereas, post-transcriptional regulation involves multiple mechanisms including regulation of mRNA stability, miRNA interaction, recruitment of the polysome or translational activators/repressors [31]. The indication for post-transcriptional regulation, more specifically potential translational regulation, was derived from TPA-induced nucleo-cytoplasmic translocation of ALOX12-AS1 in THP-1 cells, RNase protection assay and the presence of ALOX12 mRNA and ribosomal protein in ALOX12-AS1 lncRNA pulldown product. It is possible that the nucleo-cytoplasmic abundance of ALOX12-AS1 is tightly regulated by epigenetics (post-transcriptional RNA modifications) or by interacting proteins that associate with the ribonucleoprotein complex containing ALOX12-AS1. This conjecture is supported by the reduction in ALOX12 protein that occurs in response to TPA-induced nucleo-cytoplasmic translocation of ALOX12-AS1 lncRNA (FIG. 5). TPA is a PKC activator [35] and the PKC family consists of nine genes that encode different isozymes with roles in cell proliferation, apoptosis, and migration [36]. It is therefore reasonable to speculate that PKC is required in the processes that trigger translocation of ALOX12-AS1 lncRNA, even without further identification and characterization of the lncRNA associated protein, RNA and chromatin components. There are examples that substantiate this premise. For example, one PKC effector is the RNA-binding protein HuR (human antigen R) which associates with numerous transcripts, coding and noncoding, and controls their splicing, localization, stability, and translation [37]. PKCα has been demonstrated to phosphorylate HuR in the nucleus, leading to ATP-dependent HuR cytoplasmic translocation which enhances its binding to COX2/PTGS2 mRNA leading to increased mRNA stability, COX2 production, and subsequent increase in prostaglandin E2 synthesis [38]. Besides coding transcripts, HuR also has the ability to bind to and regulate the functions of lncRNAs [39, 40]. Under reduced HuR levels, a long intergenic noncoding (linc) RNA, lincRNA-p21, accumulates in human cervical carcinoma HeLa cells, increasing its association with JUNB and CTNNB1 mRNAs and selectively lowering their translation [40].

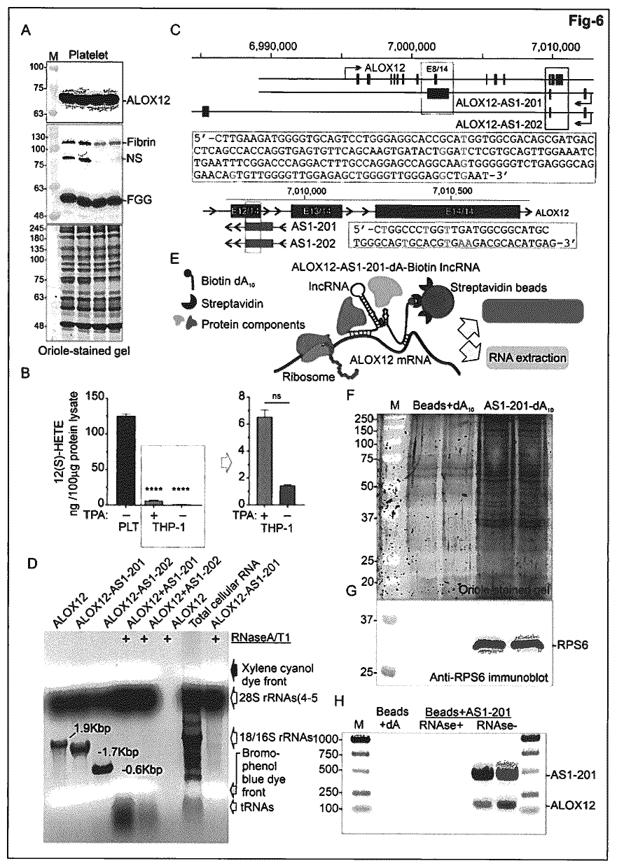

ALOX12-AS1 cytoplasmic abundance is a deterministic factor for translational downregulation of ALOX12. The functional involvement of the duplex in mediating translational interference was supported by the presence of ribosomal protein and ALOX12 mRNA in the ALOX12-AS1-201 lncRNA pulldown fraction indicating potential association of the ribosome in the duplex (FIG. 6). We used HEK293 cells for the biotin-labeled lncRNA transfection and the lncRNA pulldown assay because HEK293 is an excellent cell line to use in transfection experiments. Furthermore, ALOX12-AS1 lncRNA is expressed in HEK293 cells, and the Human Protein Atlas (HPA) RNA sequencing of mRNA extracted from unsynchronized log phase HEK293 cells detected ALOX12 mRNA which is in agreement with our finding ALOX12 mRNA in the antisense lncRNA pulldown product (FIG. 6H) [28]. The exact molecular nature of the antisense-sense translational interference needs to be investigated in the future; however, based on existing literature, some predictions can be made about the nature of this interference. Interestingly, the sense-antisense overlapping segments in the ALOX12-AS1 lncRNA isoforms are immediately adjacent to an SVA-retroposon/short interspersed element (SINE) and a long interspersed element (LINE), respectively. RNAs transcribed from SINEs repress transcription of some protein-encoding genes through binding to RNA polymerase II [46]. Interestingly, a classic antisense lncRNA-mediated cis-regulation (translational) study by Carrier et al. [47] showed that two embedded SINE elements (SINE-B1 and -B2) in the non-overlapping part of ubiquitin carboxy-terminal hydrolase L1 antisense lncRNA (Uchl1os) are instrumental in enhanced recruitment of the polysome to Uchl1 mRNA under rapamycin treatment-induced stress conditions resulting in an increase in Uchl1 protein levels [47]. Carrier et al also showed that under stress conditions the predominantly nuclear-localized Uchl1os translocate to the cytoplasm to exert its action [47]. In a contrasting study involving lncRNA-mediated trans-regulation, human lncRNA-p21[48] was demonstrated to repress the translation of JUNB and CTNNB1 (β-catenin) mRNAs by recruiting translational repressors [40]. Thus, there are examples of both translational induction or repression of target genes by antisense lncRNAs. Unlike mRNAs, many lncRNAs have nuclear residence with a focal or dispersed localization pattern (NEAT1) [49], however, others were found either in both the nucleus and cytosol (TUG1, HOTAIR) or exclusively in the cytosol (DANCER) [50]. The lncRNA-p21 was found to be more abundant in the cytosol compared to the nucleus [40]. Thus, a high cytosolic abundance of the antisense lncRNA appears to be an important determinant for translational interference. Further, the 7SL-RNA SINE (Alu) element [51] embedded in proximity of the sense-antisense overlapping region in the ALOX12-AS1 lncRNA is part of a signal recognition particle (SRP). The SRP is a cytosolic ribonucleoprotein complex that couples the synthesis of nascent proteins to their proper membrane localization [52] and guides secretory proteins to biological membranes in all organisms [53].

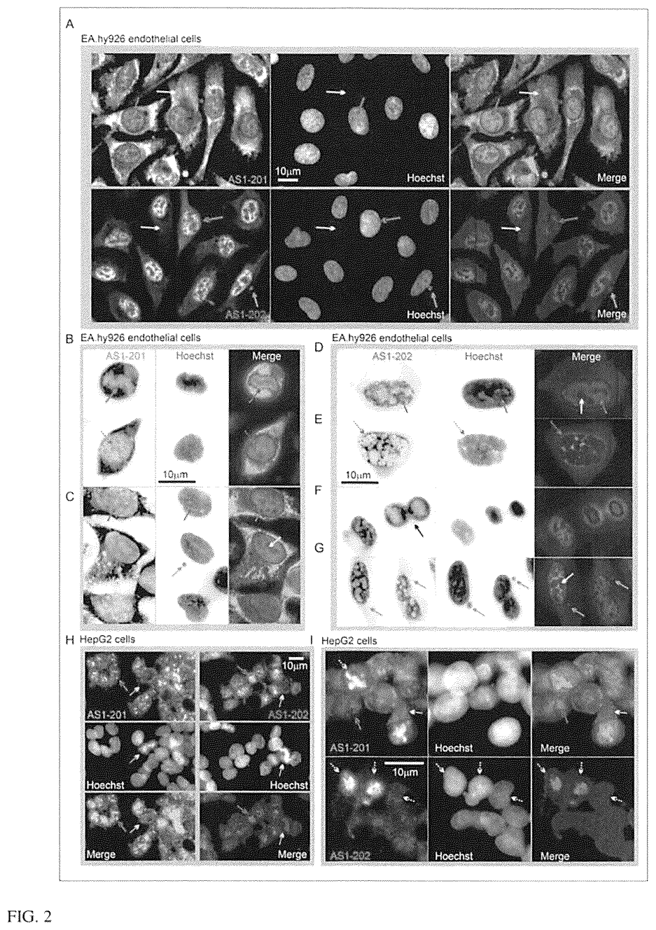

Our extensive RNA-FISH and RT-PCR-based study provides comprehensive insight into the tissue-specific expression and cell-type-specific differential intracellular distribution of ALOX12-AS1 lncRNA isoforms. The ALOX12-AS1 lncRNA was found in different adult human tissues as well as in all five human cell lines tested. This indicates that ALOX12-AS1 genes are ubiquitously expressed, and this conclusion is supported by the GTEx human gene expression studies [54]. Multiple transcriptome profiling studies have shown that few lncRNAs are ubiquitously present across all tissue or cell types [55] and those that are ubiquitously present are often highly abundant. In contrast, lncRNAs present in one tissue or cell type tend to be expressed at low levels [56]. These suggest that ALOX12-AS1 lncRNA may have an important housekeeping function in mammalian cells in different subcellular compartments involving cytosol, nucleolus, and nuclear speckles. Another important observation in our RNA-FISH study is that ALOX12-AS1 lncRNA exhibited differential abundance during cell division, specifically during metaphase, depending on cell type (FIG. 2). Multiple factors, such as a specific RNA motif [57] or RNA-protein assemblies may dictate the subcellular localization of lncRNAs and define their function [58].

The in vitro cell-free protein extract-based ALOX12 enzymatic activity assay revealed that the lncRNA-mediated regulation of ALOX12 protein level may have functional consequences and, therefore, be biologically relevant. Though we have not compared the relative abundance of ALOX12 protein level between platelets and other cell-types, a relatively high level of ALOX12 protein has been reported in rat platelets compared to different transformed rodent (3LL and W256) and human cancer (A431 and HEL) cell types. Therefore, it is expected that high ALOX12 in the platelets would translate to a higher enzymatic conversion of arachidonate to 12S-HETE which is reflected in our ELISA-based study. Platelet-mediated catalysis of arachidonic acid to 12S-HETE has been previously reported, justifying our use of platelet lysates as a positive control for ALOX12 enzymatic activity [59, 60]. The ELISA-based detection of 12S-HETE in arachidonate-treated THP-1 and MLC cell lysates supports our previous findings involving the metabolism of α-linolenic acid in THP-1 and TPA-induced MLC cells and subsequent detection of arachidonate-derived oxylipins including 12S-HETE using high pressure liquid chromatography (HPLC) with tandem mass spectrometry (MS-MS) [61].

Validation of research antibodies is an important issue [62]. Well-characterized antibody reagents play a key role in the reproducibility of research findings [63]. We used three anti-ALOX12 antibodies including two rabbit polyclonal antibodies raised against different N (186-231 aa) and C-terminal (609-628 aa) epitopes and one mouse monoclonal antibody raised using overlapping N-terminal epitopes (131-230 aa). The antibodies exhibited a considerable difference in their ability to detect different species of ALOX12 including native versus recombinant proteins in a cell-type-specific manner. This difference may be due to the recognition of different conformations (PTM forms) in a cell-type-specific manner. High throughput mass spectrometric analysis detected phosphorylation of ALOX12 at multiple serine, threonine, and tyrosine residues including S244 and S246 residues as archived in the PhosphositePlus database [64, 65]. ALOX12 phosphorylation (Y19 and Y614)-dependent modulation of its enzymatic activity has been demonstrated in an overexpression-based study using HEK293 [43]. The S244/246 residues are in close proximity to the rabbit/mouse ALOX12 N-terminal epitope. Phosphorylation of ALOX5 at multiple residues has been reported [66] that significantly alters molecular structure [67]. Therefore, it is conceivable that the cell-type-specific phosphorylation of ALOX12 in these residues may induce a conformational change in ALOX12 affecting antibody-based detection. The conformation-specificity of antibodies is an inherent limitation of immuno-detection methods and is highlighted in a recent study [68] that showed that an antibody raised against P-Tyr307 residue of protein phosphatase 2A catalytic subunit (PP2Ac) that has been extensively used in several studies, is sensitive to additional protein modifications that occur near Tyr307, including Thr304 phosphorylation and Leu309 methylation, when these PTMs are present. Thus, it has been suggested that the studies that used these antibodies to report PP2Ac hyper-phosphorylation require reinterpretation, as these antibodies cannot be reliably used as readouts for a single PP2Ac PTM change. Besides, our preliminary mass spectrometric analysis of ALOX12 identified a Lysine 217-E-Gly-Gly modification (data deposited in the Global Proteome Machine database, identification number: GPM10000003125) that is typically generated following trypsin digestion of ubiquitinated proteins [69]. Therefore, the higher molecular weight fractions of ALOX12 may represent PTMs that imparted additional mass, for example, ubiquitination. Thus, our study followed the guidelines of standard immunoblotting-based detection [70] and sets a high standard. Besides, this study also provides insight into the complexity of antibody-based detection of ALOX12 and sets the direction for future studies to identify the PTMs of ALOX12 and their functional relevance, which is lacking in the existing literature. Moreover, the above deliberation may provide an interpretation to the difference between our study vs other studies [41-45] in terms of the presence or absence of ALOX12 protein in HEK293 cells.

The biological relevance of the phorbol-ester (TPA) induced lncRNA transcytosis and ALOX12 regulation is an important aspect of this study. Murine 8S-lipoxygenase is a TPA inducible lipoxygenase [71] and genetic deletion of murine 12R-lipoxygenase resulted in ichthyosis, a cornified skin phenotype [72]. TPA treatment as well as epidermal growth factor (EGF)-PKC signaling increased the expression of ALOX12 mRNA in a time-dependent manner in skin epidermal carcinoma-derived A431 cells [73, 74]. Moreover, alteration in PKC activity has been suggested to underlie the pathophysiology of psoriasis, a hyperplastic skin disorder [75]. The human protein atlas database documented high-level ALOX12 protein expression in skin epidermal cells [28]. Taking together all this information it is possible to realize that our observations of the lack of ALOX12-AS1 lncRNA expression in primary human skin tissue vs expression of the lncRNA in transformed A431 cells and the abundance of ALOX12 proteins in both skin and A431 cells as well as TPA induced increase of ALOX12 mRNA expression in A431 cells makes these tissue/cell-based system an ideal model for further study of the biological relevance of lncRNA mediated regulation of ALOX12 and its potential therapeutic exploitation in the future. Similarly, the significance of the TPA-induced lncRNA transcytosis and ALOX12 regulation cannot be ignored. This is relevant due to the observation that TPA-induced adhesion of cancer cells/neutrophils to endothelial cells along with concomitant production of lipoxygenase catalytic products (LTB4 and 5-HETE) has been reported and this phenomenon was inhibited by lipoxygenase inhibitors [76-78]. Besides, a TPA-PKC pathway-dependent and 12S-HETE-stimulated expression of the cell surface integrin in vascular endothelial cells was identified as a mechanism for cell adhesion and expansion [79]. ALOX12 mRNA expression has been reported in HepG2 cells and treatment of HepG2 cells [80] with elaidic acid, the most abundant trans fatty acid, led to a 1.8 fold reduction of ALOX12 mRNA expression suggesting a potential link between fatty acid metabolism and ALOX12 [81]. Thus, apart from the existing model of the regulation of bioactive lipid mediators, our findings of a lncRNA-mediated downregulation of ALOX12 may serve as an alternative cell-type-specific mechanism in a tissue-specific context.

According to an aspect of the invention, there is provided a method for treating a disease or disorder associated with Arachidonate 12-lipoxygenase (ALOX12) protein levels, said method comprising:
administering an effective amount of an ALOX12-AS1 modulating compound to an individual in need of such treatment, the effective amount altering ALOX12 mRNA-ALOX12-AS1 lncRNA interaction and thereby altering ALOX12 protein levels compared to an untreated control.

The ALOX12-AS1 modulating compound may promote or enhance or increase nucleo-cytoplasmic translocation of ALOX12-AS1 lncRNA.

The ALOX12-AS1 modulating compound may be a protein kinase C inhibitor, for example, a phorbol ester.

The ALOX12-AS1 modulating compound may be an ALOX12-AS1 lncRNA antisense oligonucleotide.

The disease or disorder may be selected from the group consisting of: cardiovascular disease, a skin disorder, a skin inflammatory disease, non-alcoholic fatty liver disease, psoriasis, diabetic nephropathy, platelet agglutination, cancer and Alzheimer's disease.

While not wishing to be bound or limited to or by a particular theory or hypothesis, the inventor believes that an equimolar ratio of the lncRNA:ALOX12 mRNA in the cytoplasm is required for the maintenance of basal ALOX12 protein level. Accordingly, inhibition of ALOX12 mRNA by this mechanism is not “all or nothing” but rather may be considered to exist as a continuum that is influenced by changes in ALOX12-AS1 lncRNA concentration in the cytoplasm. Specifically, it is believed that the ratio of the lncRNA:ALOX12 mRNA is important. If an equimolar ratio is maintained then a basal ALOX12 protein level is maintained. If the ratio is disturbed by any means, for example, TPA induced nucleo-cytoplasmic translocation or ASO-mediated degradation of the lncRNA, then ALOX12 protein level is affected. In some embodiments, the antisense oligonucleotides are 15-25 nucleotide long DNA sequences. ASO sequences are modified for increased stability and binding affinity.

The most widely used modifications are phosphorothioate (PS) bonds, which are added throughout the oligonucleotide backbone to provide nuclease resistance. PS bonds have the potential to increase toxicity, which can be minimized by including an additional 2′-O-Methyl (2′OMe) RNA to create a “gapmer” ASO. DNA with these modifications display more nuclease resistance, lower toxicity, and increased hybridization affinities within cells and in vivo. Some examples of suitable ASOs include but are by no means limited to:

Antisense Oligonucleotides (ASO): Gapmers
(SEQ ID NO: 3)
mA*mG*mU*mG*mA*A*G*G*G*A*G*G*A*A*A*mU*mG*mG*mU*mA
(SEQ ID NO: 4)
mG*mU*mA*mU*mG*T*T*A*G*C*A*T*G*G*T*mA*mA*mU*mG*mA
(SEQ ID NO: 5)
mG*mU*mA*mC*mU*T*A*T*T*A*A*T*C*T*A*mU*mU*mA*mA*mA
(SEQ ID NO: 5)
mG*mU*mA*mC*mU*T*A*T*T*A*A*T*C*T*A*mU*mU*mA*mA*mA
(SEQ ID NO: 6)
mA*mG*mA*mA*mC*T*G*T*C*A*A*T*A*A*T*mC*mU*mU*mA*mC
(SEQ ID NO: 7)
mG*mG*mA*mA*mU*G*G*T*T*A*C*A*A*A*A*mU*mA*mA*mU*mG
(SEQ ID NO: 8)
mG*mU*mA*mA*mG*A*T*T*T*T*C*C*A*C*A*mA*mC*mA*mA*mG
(SEQ ID NO: 9)
mU*mA*mA*mG*mA*A*G*T*A*G*A*T*G*G*C*mC*mC*mA*mU*mU

In this table, 2-O-Methyl bases are represented with a lower case “m” in front of each base and an asterisk “*” between the bases indicates phosphorothioate bonds.

Alternatively, the lncRNA can be targeted by designing ASOs targeted to the embedded repeat elements in ALOX12-AS1 lncRNA. For example, a long interspersed element (LINE) in ALOX12-AS1-201 isoform is “TTATTGAACTCCCATTCCGTATATGACATTATAGTCAGCATTCTTGGGGAAACAG AGATGACTAAGTAAGATTATTGCCTTCTAGGAGGTTATAGTCTAGCAGGAG” (SEQ ID NO:10) and a SVA-retroposon/short interspersed element (SINE)-in the same isoform is “GAGACCGGGACTTGCTGTGTTGCCCAGGCTGGTCTTGAACTCCTGGTCTTAAGCA ATCATACTGCTTTGGCCACCCAAAGCACTGGGATTACAGGCGTGAACCACCACAC CCAGCTCTTAA” (SEQ ID NO:11).

The RNA interference via synthetic small interfering RNA (siRNA) silences the target gene by degrading the mRNA through Drosha/Dicer processing pathways which occurs in the cytoplasm. Most noncoding RNAs (lncRNA) are localized to the nucleus, limiting the use of siRNA for their silencing due to the low prevalence of the requisite enzymes (Drosha/Dicer etc) in the nucleus. However, RNase H, which is present in the nucleus, can be engaged to mediate RNA degradation, enabling the use of chemically modified antisense DNA oligonucleotides (ASO) to downregulate targeted nuclear noncoding RNAs. RNase H is non-sequence-specific endonuclease enzymes that catalyze the cleavage of RNA in an RNA/DNA substrate via a hydrolytic mechanism. Therefore, ASOs are particularly useful for silencing nuclear lncRNAs, because ASOs engage RNase H, which is prevalent in the nucleus and binds and cleaves DNA/RNA hetero-duplexes. Thus, when ASOs are used as a therapeutic means, it will go inside the nucleus and bind to the ALOX12-AS1 lncRNA creating a DNA/RNA hybrid which be degraded by RNase H and thus, it will then shift the balance of cellular lncRNA: ALOX12 mRNA molar ratio and would downregulate ALOX12 protein level. The ASO mediated downregulation is different from drug induced down regulation, both alters the lncRNA:ALOX12 mRNA molar ratio but in an opposite way.

As will be appreciated by one of skill in the art, in those embodiments in which the ALOX12-AS1 modulating compound is a small RNA molecule, compounds such as these can get inside the cell by at least two pathways:
1. Cell-surface ASO adsorption: Different cell surface proteins, including receptors, can interact with ASOs modified with phosphorothioate (PS) linkages (PS-ASO) either directly or via ligands conjugated to PS-ASOs. Cell-surface proteins can direct the internationalization of associated ASOs via clathrin- or caveolin-dependent endocytic pathways or via non-conventional endocytic pathways. Other proteins may also mediate ASO adsorption and internalization via different pathways.
2. PS-ASOs can enter cells via different endocytic pathways including macropinocytosis. Internalized ASOs can traffic from early endosomes (EE) to late endosomes (LE) and to lysosomes. ASOs must escape from endosomal organelles to reach the cytosol and nucleus to act on target RNAs.

As will be apparent to those of skill in the art, when the ALOX12-AS1 modulating compound is for example an ASO, the administration can be systemic or targeted. In targeted approach, the ASOs can be for example ligated to a specific ligand that is bound by receptors that are specifically located on the target cells. In contrast, if the modulating compound is for example a PKC inhibitor, the administration is systemic or targeted, depending on the target organ/tissue. For example, if the target organ is skin, then a targeted delivery of the drug to skin in the form of skin ointment or formulated as a skin ointment will serve the purpose. Other suitable delivery methods will be apparent to those of skill in the art and are within the scope of the invention.

As discussed herein, the ALOX12-AS1 modulating compounds of the invention can be used for the treatment of any disease or disorder associated with abnormal expression of ALOX12 protein. Suitable diseases and/or disorders include but are by no means limited to acute or chronic inflammatory disease, for example atherosclerosis, obesity and the like. In addition, suitable compounds can be used as an anti-platelet therapy for the deregulation of ALOX12 in the platelet. As discussed herein, these compounds can also be used for reducing skin inflammatory diseases, more specifically, Psoriasis as ALOX12 is highly expressed in skin epidermal cells.

A method for determining if a patient is a candidate for further screening for a disease characterized by abnormal ALOX12 protein levels comprising:
measuring expression levels of ALOX-AS1 lncRNA and a reference lncRNA in a blood sample taken from the patient;
comparing the patient ALOX12-AS1 lncRNA expression level to a control ALOX12-AS1 lncRNA expression level, thereby providing an ALOX12-AS1 lncRNA ratio;
comparing the patient reference lncRNA expression level and to a control reference lncRNA expression level, thereby providing a reference lncRNA ratio, wherein
if the ALOX12-AS1 lncRNA ratio is greater than the reference lncRNA ratio, the individual is screen for an acute or chronic inflammatory disease
if the ALOX12-AS1 lncRNA ratio is lower than the reference lncRNA ratio, the individual is screened for a decreased inflammatory response disease.

In some embodiments, the lncRNA is screened in the blood as a serum or exosome-based biomarker for chronic inflammatory diseases (for example, cardiovascular diseases including atherosclerosis) and malignancies. The serum/Exosome RNA will be extracted and then the lncRNA will be quantified using RT-PCR and compared with the same serum/exosomal housekeeping reference lncRNA, for example, RNA Component Of Signal Recognition Particle 7SL1 (RN7SL1), Metastasis Associated Lung Adenocarcinoma Transcript 1 (MALAT1), H19 Imprinted Maternally Expressed Transcript (H19), and Taurine Up-Regulated 1 (TUG1).

If elevated or decreased ALOX12-AS1 lncRNA is detected in the serum/exosomes, then the subject may go for a detailed screening for signs of for example Atherosclerosis or Cancer, as discussed herein.

According to another aspect of the invention, there is provided a method for determining if a compound of interest is an ALOX12 modulating compound comprising:
(a) growing a culture of cells in the presence of a compound of interest;
(b) after a suitable growth interval, measuring ALOX12 protein levels in the cells of the culture;
(c) comparing the ALOX12 protein levels to control ALOX12 protein levels from a culture of similar cells grown under similar growth conditions except for presence of the compound of interest, wherein
(d) if ALOX12 protein levels are elevated compared to the control ALOX12 protein levels, the compound is an ALOX12 activator; or
(e) if ALOX12 protein levels are reduced compared to the control ALOX12 protein levels, the compound is an ALOX12 inhibitor.

As will be apparent to one of skill in the art, ALOX12 activation/upregulation/downregulation can be detected by any of a variety of means known in the art. For example, one method is the measuring of ALOX12 lipoxygenase activity which has been demonstrated in FIG. 6. In such an assay, the drug treated cells or cell-lysates will be incubated with arachidonic acid, a substrate for ALOX12, and then the lipoxygenase activity will be measured in terms of measuring the enzymatic reaction product (12S)-hydroperoxyeicosatetraenoate/(12S)-HPETE. Other suitable methods will be apparent to one of skill in the art and are within the scope of the invention.

The compound identified in step (d) may be characterized for ALOX12-AS1 lncRNA nucleo-cytoplasm translocation activation.

The compound identified in step (e) may be characterized for ALOX12-AS1 lncRNA nucleo-cytoplasm translocation inhibition.

In some embodiments, the ALOX12-AS1 modulating compound, for example, a PKA inhibitor or activator drug can be screened for ALOX12-AS1-mediated down-regulation or upregulation of ALOX12. As will be appreciated by one of skill in the art, initially, it is not necessary to measure lncRNA nucleo-cytoplasmic or reverse translocation during the assay because such translocation will eventually be manifested in increased/decreased ALOX12 protein turnover. Therefore, measurement of the ALOX12 protein levels will be a parameter for drug selection.

However, the fact that the ALOX12-AS1 modulating compound acts through nucleo-cytoplasmic transport is important because other inhibitory mechanisms that do not involve AS1 may have undesirable side effects. For example, ALOX12 can be inhibited by a small molecule Baicalein 5,6,7-Trihydroxyflavone, known as baicalein [111], known for its anti-inflammatory properties [112, 113], however such inhibition is not selective for ALOX12 as it inhibits other ALOXs which is not desirable and may have toxic side effects [114]. Therefore, our ASO-mediated approach would provide a better alternative to selectively regulate ALOX12 through the lncRNA.

Such drugs will be useful for the diseases which require upregulation or downregulation of the inflammatory pathway. Increased ALOX12 will enhance inflammatory response and decreased ALOX12 will reduce inflammatory response thorough increased/decreased production of bioactive lipids. PKA is activated by cAMP pathway, therefore, drugs that increase/decrease intracellular cAMP or cAMP analogs will be effective in PKA-lncRNA-mediated regulation of ALOX12.

The invention will now be further explained and/or elucidated by way of examples; however, the invention is not necessarily limited to or by the examples.

### Example 1—Expression of ALOX-12-AS1 lncRNA Isoforms in Adult Human Tissues

Based on the Ensemble genome database (version GRCh38/hg38), the ALOX12 gene is located on the sense (+) strand of chr17: 6,994,196-7,010,754, whereas the overlapping ALOX12-AS1 is localized on the antisense (−) strand of chr17: 6,876,635-7,012,349 (FIG. 1A) [23]. Fifteen transcripts of ALOX12-AS1 have been mentioned in the Ensemble database (version GRCh38/hg38), but only nine transcripts successively numbered as 201-209 are supported by at least one Expression Sequence Tag (EST). Therefore, we considered those nine transcripts for validation and their exon-intron structures are summarized in FIG. 1A. We used primary adult human adipose, artery, bone marrow, cerebellum, cortex, intestine, liver, smooth muscle, and skin tissues to characterize tissue-specific expression of ALOX12-AS1 isoforms by RT-PCR using isoform-specific primer sets (Table 1). We initially designed nine sets of primers, each specific to one isoform (Table-1), but due to the degree of overlap between the isoforms, we finally standardized the procedure using one forward primer and four reverse primers to amplify all nine isoforms and generate amplicons of variable lengths specific to each isoform that could be distinguished by agarose gel electrophoresis (Table 1 and FIG. 1A). The expected lengths of the isoform-specific amplicons are mentioned in Table-1. Singleplex RT-PCR amplification using cDNAs equivalent to 100 ng total RNA revealed the presence of bands corresponding to different ALOX12-AS1 isoforms in a tissue-specific manner (FIG. 1B-E), which is summarized in FIG. 1F. Interestingly, ALOX12-AS1-201 and -202 isoforms are the predominant transcripts and abundantly expressed in all examined tissues except cerebellum and skin. The absence of ALOX12-AS1 in the cerebellum may be due to receiving RNA of low-quality from a commercial source as evidenced by the lack of amplification of GAPDH. In contrast, ALOX12-AS1 may not be expressed in the skin, since GAPDH amplification was detected. The PCR products corresponding to ALOX12-AS1-201 and -202 amplicons were verified by Sanger DNA sequencing. These findings also support the presence of the lncRNAs in 53 human tissues analyzed by RNA sequencing and human gene expression array-based studies via the Genotype-Tissue Expression (GTEx) Project [54]. The GTEx project revealed that ALOX12-AS1-201 is the predominant isoform in the majority of the human tissues, whereas ALOX12-AS1-202 is more abundant in the human brain. These findings are in agreement with the observations reported here.

### Example 2—Expression of ALOX12-AS1 lncRNA Isoforms in Five Human Cell Lines

Based on the tissue-specific expression of ALOX12-AS1 isoforms, five transformed human cell lines were selected to further examine cell-type-specific expression of the ALOX12-AS1-201/202 lncRNAs: A431 (skin epithelial [83]), EA.Hy926 (endothelial [84]), THP-1 (monocyte [85]), HEK293 (human embryonic kidney [86]) and HepG2 (hepatoma [87]). In all five cell lines, Northern blotting using isoform-specific fluorescently labeled RNA probes revealed the presence of ˜1.8 kb and 0.7 kb full-length transcripts corresponding to the previously reported ALOX12-AS1-201 and -202 lncRNA isoforms, respectively (FIG. 1G). It is important to note that the ˜2 kb cDNA sequence submitted with a GeneBank identifier AK074580.1 [26] corresponding to ALOX12-AS1-201 isoform has a 290 nucleotide sequence element at the 5′ end that did not match to the ALOX locus and, therefore, it may be considered a cloning artifact. Thus, our observation of a ˜1.8 kb band matched the anticipated length of the ALOX-12-AS1-201 lncRNA. ALOX12-AS1-201 and -202 lncRNA-specific primers were used to study the expression of the isoforms in five cell lines (Table 1). The cDNAs were prepared using both random hexamers and oligo-dT as a means of examining the polyadenylation status of ALOX12-AS1 lncRNAs, since non-polyadenylated RNAs will not be amplified in PCR when oligo-dT is used for first-strand cDNA synthesis. The ALOX12-AS1-201/202 isoform-specific primers generated the expected PCR products (494/496 bp, respectively) in all five cell lines, thus indicating the expression of poly-A tailed lncRNAs. The presence of some low/high molecular weight shadow bands with the expected amplicons may represent other isoforms co-amplified or PCR artifacts. The ALOX12-AS1-202 isoform was expressed at relatively lower levels in A431 cells compared to the EA.hy926 and THP-1 cells. qPCR revealed a significantly reduced copy number of ALOX12-AS1-202 compared to ALOX12-AS1-201 in all cell lines examined except THP-1 where it was increased significantly.

### Example 3—Detection of ALOX-12-AS1 lncRNA Isoforms in Primary Adult Human Tissues by RNA Fluorescence In Situ Hybridization (RNA-FISH)

RNA-FISH using fluorescent-labeled probes revealed the presence and intracellular localization of ALOX12-AS1-201/202 lncRNAs in adipose, PBMs and liver tissues. High-magnification images revealed both cytosolic and nuclear localization of the lncRNA isoforms in the different tissues. The RNA-FISH wide-field tissue-based images showed the extent of cell-type-specific expression and intracellular localization within each tissue, as discussed herein. With respect to their presence in the nucleus, both lncRNAs appeared to be primarily in the nucleolus and nuclear speckles in a tissue-specific manner. Interestingly, both lncRNAs were predominantly in the cytosol in a fraction of PBMs exhibiting intense Hoechst staining. This staining pattern may be due to the state of the chromatin, which appears condensed and thus may be indicative of apoptotic cells.

The tissue and cell-type/organelle-specific localization of ALOX12-AS1 lncRNA validated the effectiveness of the FISH probes. In addition, two different experimental approaches were used to further validate the FISH probes. First, adult human abdominal skin tissue which tested negative for RT-PCR based amplification of ALOX12-AS1 specific products (FIG. 1B) served as a negative control since no signal was detected using ALOX12-AS1-201/202-specific FISH probes. Second, we used T7 RNA polymerase to generate sense probes corresponding to ALOX12-AS1-201/202 FISH probes (generated by Sp⁶ RNA polymerase) and performed FISH using HEK293 cells. As anticipated, the sense probes were unable to generate any detectable signal, thereby providing additional validation of the specificity of the FISH probes used in this study.

### Example 4—RNA-FISH Indicates Cell-Type-Specific Differential Intracellular Localization of ALOX12-AS1 lncRNA Isoforms

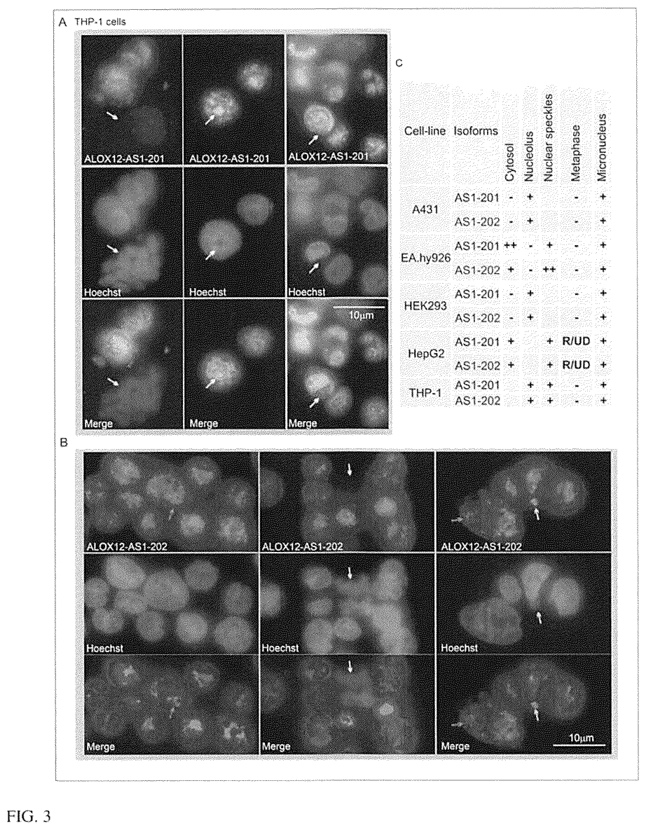

RNA-FISH was performed to determine the subcellular localization of ALOX12-AS1-201/202 isoforms in A431 (Fig S8A-C), HEK293 (FIG. 1H), EA.hy926 (FIG. 2A-G), HepG2 (FIG. 2HI) and THP-1 (FIG. 3AB) cells. The ALOX12-AS1 lncRNA isoforms exhibited cell-type-specific differences in their intracellular localization, which is summarized in FIG. 3C. Both lncRNA isoforms were predominantly nuclear in all cell types (FIG. 1H, 2, 3) and cytosolic localization was prominent in EA.hy.926 cells (FIG. 2A). Higher magnification images revealed prominent nucleolar localization of both lncRNA isoforms in the A431 and HEK293 cells. A small fraction of THP-1 cells also exhibited potential nucleolar localization (FIG. 3A). Simultaneous co-hybridization using differentially labeled RNA-FISH probes revealed a complete overlap of the lncRNA isoforms in the granular region of the nucleoli of both cell types suggesting potential localization in the fibrillar centers and/or dense fibrillary components of the nucleolus. Unfortunately, the RNA-FISH sample preparation technique destroyed the epitopes of the most commonly used nucleolar-specific markers, NCL (Nucleolin) and UTP6 (U3 Small Nucleolar RNA-Associated Protein 6 Homolog), making them unsuitable for co-immunofluorescence. Therefore, the nucleolar localization of ALOX12-AS1 lncRNA was resolved on the basis of two separate experiments. The first visualized the nucleolar structures under bright field microscopy after transfection with GFP-tagged NCL and compared this information with the RNA-FISH results. The other method employed subcellular fractionation to isolate the nucleoli and is described in the next section. The nucleolus of HEK293 cells appeared as discernible distinct-textured structures within the nucleus in phase-contrast bright-field microscopic images while also exhibiting the presence of GFP-NCL. Similar nucleolar structures in A431 nuclei were found positive for ALOX12-AS1-201/202 RNA-FISH signals. The RNA-FISH probe signals for both lncRNA isoforms were undetectable in metaphase of A431 and HEK293 cells (FIG. 1H), but in EA.hy926 cells, the lncRNAs appeared in the interchromatid regions of the condensed chromosome in early metaphase cells (FIG. 2E) as well as in the cytosol in anaphase stage cells (FIG. 2F) indicating that the subcellular distribution of these lncRNAs are dynamically controlled during cell division.

### Example 5—Subcellular Fractionation Shows Predominantly Nuclear Localization of the ALOX12-AS1 Isoforms in Different Cell Types

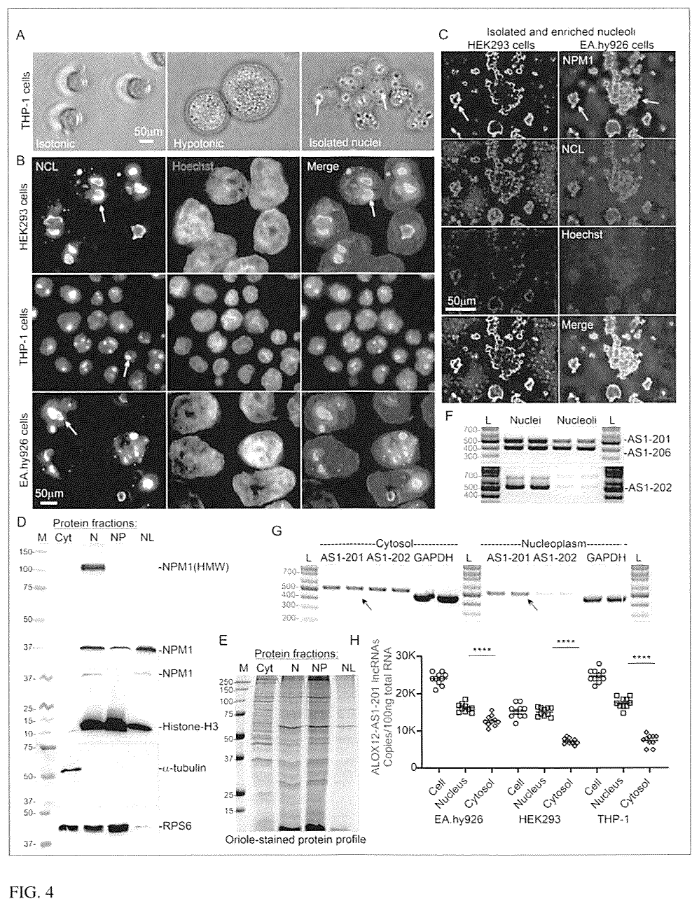

We performed subcellular fractionation to study the nucleo-cytoplasmic distribution of the ALOX12-AS1 lncRNAs. The nucleoli in adherent cells are clearly visible in bright-field images but not in suspension cells, for example THP-1 cells (FIG. 4A). Exposure to the hypotonic medium during nucleolar fractionation process swelled THP-1 cells and subsequent homogenization made nucleolar structures visible in isolated nuclei under bright-field microscopic observation (FIG. 4A). Immunofluorescence using a nucleolus-specific marker, nucleolin (NCL), revealed distinct nucleolar structures in HEK293, THP-1 and EA.hy926 cells (FIG. 4B) which were isolated and enriched by subcellular fractionation (FIG. 4C).

Immunofluorescence using the nucleolus-specific markers, NCL and nucleophosmin (NPM1), revealed an effective enrichment of the nucleoli (FIG. 4C). Immunoblotting with protein markers for cytosol (α-tubulin and Ribosomal Protein S6—RPS6), nucleolus (NPM1), and nucleus/nucleoplasm (Histone H3/RPS6) was used to validate the subcellular fractions (FIG. 4D). Immunoblotting revealed a relatively high abundance of NPM1 and less abundance of histone/RPS6 in the nucleolar fractions compared to the nuclear/nucleoplasm fractions, indicating high enrichment of nucleoli during the purification process (FIG. 4D). The presence of α-tubulin and RPS6 and the absence of Histone H3 in the cytosol further indicated the purity of the cytosolic fraction. The effectiveness of the fractionation process was further established by the distinct fingerprint of the total protein band profile in an oriole-stained gel (FIG. 4E). Total RNA was extracted from the cytosol, nucleus, nucleoplasm (nucleus-nucleoli) and nucleolar fractions and RT-PCR was performed using isoform-specific primers to further demonstrate subcellular organelle specific localization of ALOX12-AS1 lncRNAs in EA.hy926 cells (FIG. 4FG). RT-PCR using cDNA prepared from the different subcellular RNA fractions revealed the presence of ALOX12-AS1-201 and 202 isoforms in the cytosol, nucleoplasm, and nucleolus of the EA.hy926 endothelial cells (FIG. 4FG), which corroborated the findings obtained with RNA-FISH. Quantitative RT-PCR revealed a significantly lower cytosolic abundance of ALOX12-AS1 compared to the nuclear abundance in EA.hy926, HEK293 and THP-1 cells (FIG. 4H).

### Example 6—Characterization of ALOX12 Protein Levels in Different Transformed Human Cell Lines

To see if antisense ALOX12-AS1 lncRNA regulates the expression of ALOX12, ALOX12 protein levels were characterized using two N-terminal (N) and one C-terminal (C) epitope-based anti-ALOX12 antibodies (Table 1). The theoretical molecular mass of the consensus ALOX12 protein (ID: ENSP00000251535) is 75.6 kDa as calculated by the ExPASy Compute pI/MW tool [88]. The anti-ALOX12-(N) and -(C) antibodies recognized a 75 kDa Flag-tagged ALOX12 protein transiently expressed in HEK293 cells. The rabbit polyclonal anti-ALOX12-(N) antibody recognized a 110 kDa protein in EA.hy926, A431, and THP-1 cells. This band was less abundant in THP-1 and EA.hy926 cells compared to A431 cells. The possibility that a transcriptional isoform is responsible for the appearance of the p60 kDa ALOX12 in THP-1 cells was verified by performing a long-range amplification of the full-length ALOXs cDNAs in A431 and THP-1 cells. Long-range PCR revealed the amplification of ALOXE3, ALOX5, ALOX12, and ALOX12B cDNAs of expected amplicon lengths, specifically in the case of ALOX12 where the expected amplicon size (1992 nucleotides) matched to the observed 1.9 kb band. Interestingly, a shorter ˜1500 nucleotide long amplicon was observed in THP-1 cells along with the ALOX5 and ALOX12 amplicons of expected lengths. This result indicates the possibility of the existence of a shorter transcriptional isoform of ALOX12 in THP-1 cells which may be responsible for the appearance of p60 kDa ALOX12 in THP-1 cells. Furthermore, recombinant Flag-ALOXs were overexpressed in HEK293 cells to check the specificity of anti-ALOX antibodies. The anti-Flag antibody detected all recombinant Flag-ALOX proteins at around 75 kDa. Based on these results, we used the anti-ALOX12-(C) antibody in subsequent experiments. Overall, these data indicate differential expression of native ALOX12 proteins and some higher/lower molecular weight fractions in the five cell lines tested.

### Example 7—Phorbol Ester Treatment of THP-1 Cells Suppresses ALOX12 Protein Level by Inducing Nucleo-Cytoplasmic Translocation of ALOX12-AS1

Phorbol ester (12-O-Tetradecanoylphorbol 13-acetate: TPA) is an activator of protein kinase C (PKC) that has been frequently used for inducing the differentiation of different cell types, including myeloid cells [35]. Activation of human monocytic cells affects ALOX expression [89, 90]. Therefore, we induced differentiation of monocytic THP-1 cells to macrophage-like cells (MLCs) with 100 nM TPA [91, 92] and monitored the ALOX12 protein level as well as nucleo-cytoplasmic translocation of ALOX12-AS1-201. Over the TPA treatment period (1-72 h), ALOX12 protein levels were dramatically reduced (FIG. 5A). We concurrently monitored expression of the integrin subunit a M (ITGAM), also known as CD11b, which regulates myeloid cell polarization and is expressed in macrophages to facilitate adhesion, migration, chemotaxis, and accumulation during inflammation [93, 94]. Immunoblotting revealed ITGAM was expressed at 72 hours indicating complete differentiation of THP-1 cells to macrophages at this time-point (FIG. 5A). The progressive differentiation of THP-1 cells from monocytes to macrophages was further supported by immunofluorescence which revealed a gradually increasing expression and translocation of CD68 to endosomal structures during TPA-induced THP-1 cell differentiation (FIG. 5B). CD68 is a transmembrane glycoprotein that is mainly located in the endosomal/lysosomal compartment but can rapidly shuttle to the cell surface in myeloid cells [95]. Furthermore, FISH revealed that in contrast to the predominant nuclear localization of ALOX12-AS1-201 lncRNA in native THP-1 cells, TPA treatment caused translocation of the lncRNA to the cytosol in MLCs, within 1 hour of TPA treatment which correlated with a corresponding loss of ALOX12 protein at different time points (FIG. 5C). Based on these observations, we performed nuclear and cytoplasmic fractionation at 72 hours of TPA treatment. Quantitative RT-PCR using nuclear and cytosolic RNA revealed a significant increase in ALOX12-AS1-201 cytosolic abundance and a concomitant decrease in ALOX12 protein levels in TPA-differentiated MLCs compared to vehicle (DMSO) treated THP-1 cells (FIG. 5D-F). Interestingly, multiplex RT-PCR revealed a dramatic reduction of ALOX12-AS1-202 in TPA treated (72 hours) MLCs compared to vehicle treated THP-1 cells, whereas, ALOX12 AS1-201 and ALOX12 mRNA levels remained relatively unaltered. These results indicate a yet unknown mechanism governing the interrelationship of the relative abundance of the lncRNA isoforms.

### Example 8—Reduction of ALOX12 in TPA-Activated THP-1 Derived MLCs Leads to a Decrease in ALOX12 Enzyme Activity

We measured ALOX12 enzyme activity of THP-1 and TPA-induced MLC cell lysates and compared them to establish the biological relevance of lncRNA-mediated downregulation of ALOX12 protein level. We used a cell-free system to avoid any limitations of intracellular arachidonic acid uptake by an in vitro cell-based system. As well, we used a platelet-derived protein-lysate as a positive control for ALOX12 enzyme activity [59]. ALOX12 was originally discovered and characterized as an arachidonic acid peroxidizing enzyme in platelets [96]. Treatment with 50 μM arachidonic acid for 15 min at 37° C. resulted in the production of ˜124, ˜6, and ˜1.3 ng 12S-HETE by the platelet, THP-1, and MLC cell lysates, respectively. These data indicate there is more ALOX12 in platelets compared to the other transformed cell-types as reported in the literature [97]. Interestingly, the lower amounts of 12S-HETE produced by the MLC lysate compared to the THP-1 lysate (˜1.3 vs ˜6 ng) correlated with the reduced levels of ALOX12 protein in these cell-types. Overall, these results establish a functional link between the ALOX12 protein level and the reduced lipid peroxidizing activity in these different cell-types.

### Example 9—Detection of Sense ALOX12 and Antisense lncRNAs Interaction by RNase Protection Assay

Post-transcriptional control of gene function by lncRNAs is mediated through a variety of mechanisms including regulation of mRNA decay, activation of mRNA translation, sequestration of miRNA and miRNA-mediated repression, to name a few [98]. To achieve direct contact-based post-transcriptional regulation, lncRNA must physically interact with its target sequence to form an RNA duplex. The ALOX12-AS1-201/202 isoforms contain 52 nucleotide sequence motifs complementary to exon 14 of the ALOX12 gene (FIG. 6C). The ALOX12-AS1-201 isoform contains an additional 210 nucleotide motif that is complementary to exon 8 of the ALOX12 gene (FIG. 6C). An RNase protection assay [99] was used to verify the formation of an RNA duplex between the ALOX12 mRNA and the antisense ALOX12-AS1 lncRNA. This assay produced a smeared band below the bromophenol blue dye front (˜300 bp) that is resistant to RNaseA/T1 digestion, thus indicating the potential formation of an RNA duplex involving ALOX12-AS1-201/202 and ALOX12 mRNAs (FIG. 6D). The RNaseA/T1 protected fragments were in the 50-200 base pair range as was anticipated. This deduction is further justified by the appearance of an RNaseA/T1 protected ALOX12-lcnRNA duplex below that of the tRNA bands (usually 50-100 bases in length [100]) in the total cellular RNA profile (FIG. 6D). Smearing of the RNaseA/T1 protected duplex band is possibly due to incomplete digestion or overhangs or the presence of secondary structures.

### Example 10—Streptavidin-Mediated Pulldown of 3′ Biotinylated ALOX12-AS1 lncRNA Revealed Association with ALOX12 mRNA and Ribosomal Proteins in the Cytosol

A 3′-Biotin-dA₁₀ oligonucleotide ligated ALOX12-AS1-201 isoform was transfected into HEK293 cells and the transfected lncRNAs were crosslinked and pulled down using streptavidin-coated magnetic beads (FIG. 6E). The RNA and protein components were extracted from the pull-down product and analyzed by multiplex RT-PCR and immunoblotting for the presence of ALOX12 mRNA and interacting proteins, respectively (FIG. 6E). Oriole-staining revealed the presence of multiple proteins in the lncRNA pulldown product compared to the control beads+dA₁₀ pulldown product after separation by SDS-PAGE (FIG. 6F). Immunoblotting revealed the presence of ribosomal protein RPS6 in the lncRNA pulldown (FIG. 6F) indicating a potential association of the lncRNA-ALOX12 duplex with ribosomes. Multiplex RT-PCR revealed co-amplification of ALOX12 and ALOX12-AS1-201 specific PCR products indicating an association of the coding and non-coding RNAs (FIG. 6H). Overall, these results indicate the involvement of a ribonucleoprotein complex containing ALOX12, ALOX12-AS1-201 and ribosomal proteins which is a strong indication of translational interference. Identification of the lncRNA-associated proteins/RNAs by mass spectrometry/RNA sequencing will shed further insight into the detailed nature of this interference, but this is beyond the scope of the current study and thus will be addressed separately.

### Example 11—Cytosolic Abundance of ALOX12-AS1 Suppressed ALOX12 Protein Level at Post-Transcriptional Level in the HEK293 Cells

HEK293 cells were transiently transfected with plasmids expressing ALOX12-AS1-201/202 to study lncRNA-mediated suppression of ALOX12. Multiplex RT-PCR revealed relatively high expression of both lncRNA isoforms in the overexpressed cells (FIG. 6A, blue arrows); however, ALOX12 mRNA (FIG. 6B, red arrows) and protein levels (FIG. 6C) remained relatively unaltered compared to the untransfected cells. The nucleo-cytoplasmic abundance of ALOX12-AS1 lncRNA may be tightly regulated in HEK293 cells. Therefore, increased transcription of the transfected plasmids in the nucleus may not ensure higher levels of the lncRNAs in the cytoplasm, which may be a requisite for suppression of ALOX12. To overcome this limitation, HEK293 cells were transfected with in vitro transcribed ALOX12-AS1-201/202 mRNAs to ensure high cytoplasmic abundance of the antisense lncRNAs. Multiplex RT-PCR revealed the presence of relatively high levels of ALOX12-AS1-201/202 lncRNAs in the mRNA transfected HEK293 cells (FIG. 6DE). Interestingly, high abundance of the antisense lncRNAs in the cytosol did not alter the level of ALOX12 mRNA (FIG. 6F) but significantly reduced the amount of ALOX12 protein (FIG. 6GH). Subsequently, we labelled in vitro transcribed 3′-biotinylated lncRNAs with Cy3-conjugated streptavidin and transfected HEK293 cells with these constructs to visualize cytosolic localization of the lncRNAs (FIG. 6I). Both ALOX12-AS1-201/202 lncRNAs showed diffused staining throughout the cytosol and also present in discrete punctate vesicular structures in the cytoplasm (FIG. 6I, red arrows). The transfected ALOX12-AS1 mRNAs were excluded from the nucleus, suggesting potential post-transcriptional regulation of the lncRNA that regulates its nucleo-cytoplasmic abundance (FIG. 6J).

### Example 12—Interstitially Deleted Mutant ALOX12-AS1 201 lncRNA was Less Effective in Suppressing ALOX12 Level

The antisense-mediated regulation of ALOX12 may be exerted by the two overlapping sequence regions involving antisense ALOX121-AS1 201 and sense ALOX12 consisting of a 210 base-pair (bp) segment in ALOX12 exon 8 and a 52 bp segment in ALOX12 exon 12 respectively. In order to see the effect of this overlapping sequences on lncRNA-mediated ALOX12 transcriptional suppression, we generated mutant ALOX12-AS1-201 with interstitially deleted (A) 201 and 52 bp segments and then in vitro transcribed poly-A tailed (dA-10 nucleotides) AALOX12-As1-201 mRNA was transfected in HEK293 cells. Truncated AALOX12-AS-201 was found ˜3 fold less effective in reducing ALOX12 protein level compared to the full-length ALOX12-AS1-201 (71% vs 25% respectively). This indicated that the overlapping sequence element is to some extent responsible for mediating antisense-sense translational regulation.

### Example 13—ASO-Mediated Knockdown of ALOX12-AS1-201/202 Retained ALOX12 mRNA Level but Drastically Reduced ALOX12 Protein Level in HEK293 Cells

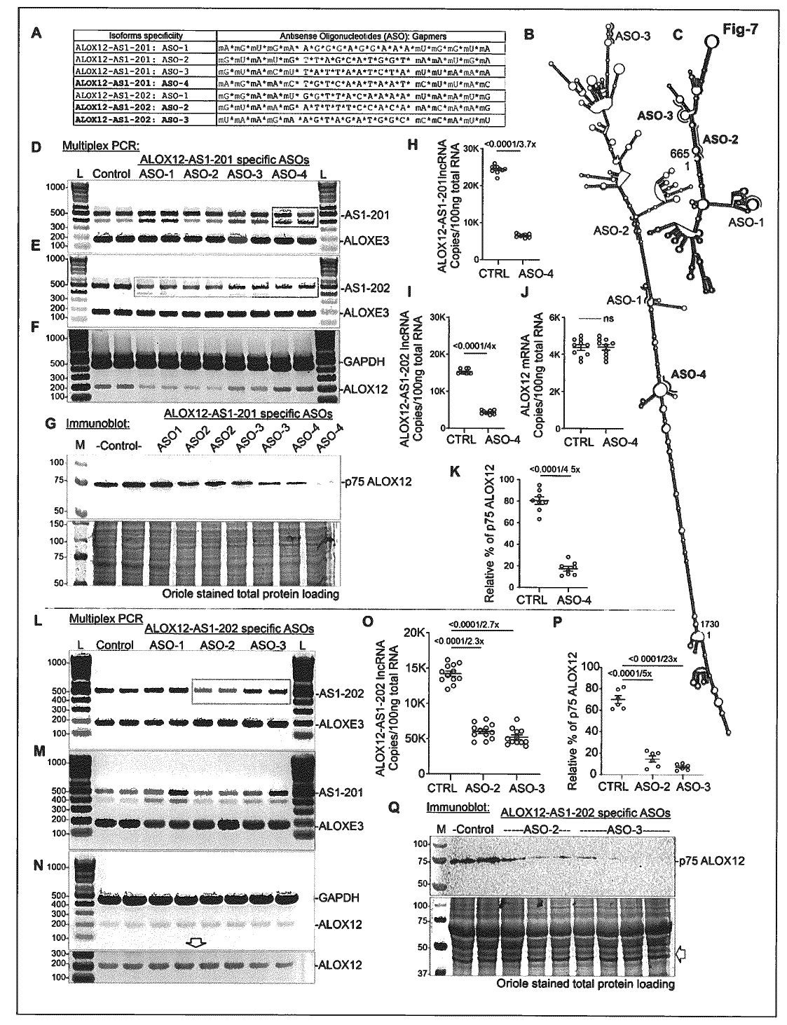

Small interfering RNA (siRNA)-mediated knockdown of ALOX12-AS1 in HEK293 cells was ineffective possibly because of the nuclear localization of the lncRNA. As an alternative approach, multiple isoform-specific oligonucleotides (ASOs) complementary to different regions of ALOX12-AS1-201/202 were designed (FIG. 7A-C). Among the four ASOs specifically targeted to ALOX12-AS1-201, ASO-4 was found to effectively knockdown (by ˜27%) ALOX12-AS1-201 within 24 hours of transfection (FIG. 7D, red rectangle). Interestingly, all four ASOs were found to have an unexpected off-target effect whereby they knocked down ALOX12-AS1-202 by 25-50% (FIG. 7E, red rectangle). While ALOX12 mRNA levels remained relatively unaltered in ASO-2/3/4 transfected cells (FIG. 7F), the ALOX12 protein level was dramatically reduced (FIG. 7J). Quantitative PCR and immunoblotting also revealed that 24 hours after transfection with ASO-4 there was a significant reduction in both ALOX12-AS2-201 and -202 isoforms (FIG. 7GH) Interestingly, while the ALOX12 mRNA level remained unchanged under these conditions (FIG. 71), the ALOX12 protein level was significantly reduced (FIG. 7K). These results further support the conclusion that antisense lncRNAs regulate ALOX12 at a post-transcriptional level.

Three ALOX12-AS1-202-specific ASOs were designed and two of them (ASO-2 and -3) reduced ALOX12-AS1-202 mRNA levels within 24 hours of transfection (FIGS. 7L&O, red rectangle). Interestingly, ALOX12-AS1-202-specific ASOs exhibited no off-target effects on ALOX12-AS1-201 isoform levels (FIG. 7M). The knockdown of ALOX12-AS1-202 by ASO-2 and -3 had no effect on the relative abundance of ALOX12 mRNA (FIG. 7N), but significantly reduced ALOX12 protein expression (FIG. 7PQ). This result again indicates that antisense lncRNA regulates ALOX12 at a post-transcriptional level.

### Example 14—Phorbol Ester Treatment of HEK293 Cells Suppressed ALOX12 Level by Nucleo-Cytoplasmic Translocation of ALOX12-AS1

Phorbol ester (12-O-Tetradecanoylphorbol 13-acetate: TPA) is an activator of protein kinase C (PKC) that has been frequently used for inducing differentiation in different cell types including myeloid cells [35]. Recently, in a separate study, we observed that during TPA-mediated differentiation of monocytic THP-1 cells to macrophage like cells, ALOX12 level is dramatically reduced (data not shown). Therefore, we treated HEK293 cells with 10 μM TPA for 1 hour and observed ALOX12-AS1-201 nucleo-cytoplasmic translocation as well as measured ALOX12 protein level. FISH revealed that TPA treatment caused dramatic nucleo-cytoplasmic translocation of ALOX12-AS1-201 compared of the vehicle (DMSO) treated cells. Subcellular fractionation and quantitative RT-PCR revealed a significant increase of cytosolic ALOX12-AS1-201 lncRNA abundance in the TPA-treated cells compared to the vehicle treatment. As anticipated, the drug-induced nucleo-cytoplasmic translocation of ALOX12-AS1-201 correlated to a significant reduction in ALOX12 protein level in TPA-treated cells compared to vehicle treatment.

### Example 15—Materials and Methods

### Adult Primary Human Tissues

The primary adult human tissue cryosections and total RNA samples were purchased from Zyagen Life Science, USA (Frozen section catalogue numbers: HF-101/103/314/413; total RNA catalogue numbers: HR-101/102/103/306/413/704/810/3014). Human blood for peripheral blood CD14⁺ monocyte (PBM) isolation was obtained by informed consent as per a protocol approved by the University of Manitoba Research Ethics Board and the St. Boniface Hospital Research Review Committee. The CD14+ PBMs were isolated via a two-step process involving preparation of peripheral blood mononuclear cells (SepMate-50 tubes, catalogue number: 85460) from which CD14⁺CD16⁻ monocytes were obtained (EasySep™ Human Monocyte Isolation Kit, Stemcell Technologies, catalogue number: 19359) according to the manufacturers' protocols [61].

### Cell Culture

A431, EA.hy926, HEK293, and HepG2 cells were grown in Dulbecco's modified Eagle's medium (DMEM) supplemented with 10% heat-inactivated FBS and 1× antibiotic-antimycotic solution (A5955, Sigma). The THP-1 cells were grown in RPMI-1640 medium supplemented with 0.05 mM 2-mercaptoethanol and 10% FBS.

RNA Extraction, cDNA Synthesis, Singleplex/Multiplex Reverse-Transcription PCR (RT-PCR) and Quantitative Real-Time PCR (qPCR)

Total RNA from different cell lines was extracted with Trizol reagent (Catalog number: 15596026; ThermoFisher Scientific, Canada) as per the manufacturer's recommended protocol. Total RNA (1-5 μg) was treated with RNase-free DNase I (Catalog number: M0303, New England Biolabs Inc., USA) at 37° C. for 15 min, subsequently heat-inactivated at 75° C. for 10 min and used for cDNA synthesis. The first-strand cDNA was synthesized using the SuperScript™ first-strand synthesis system (Catalog number: 11904018; ThermoFisher Scientific, Canada). Multiplex RT-PCR and regular RT-PCR were performed in a 20 μL reaction mix containing 1× DreamTaq buffer, 2 mM dNTP mix, 0.2 μM oligonucleotide primers, cDNAs equivalent to 100 ng total RNA, and 1.25 units DreamTaq™ Hot Start DNA Polymerase (Catalog number: EP1701; ThermoFisher Scientific) and amplified using 95° C. for 1 min, 35 cycles of 95° C. for 10 sec, 62°-65° C. for 10 sec and 72° C. for 30 sec. The RT-PCR products were separated using agarose gel electrophoresis and visualized. Absolute quantification of ALOX12-AS1 lncRNA isoforms was performed in a Mastercycler®ep realplex real-time PCR system (Eppendorf, Hamburg, Germany) using SYBR green dye (Catalog number: S7563; ThermoFisher Scientific). To determine the absolute copy number of the target transcripts, cloned plasmid DNAs containing the ALOX12-AS1201/202 isoforms were used to generate calibration curves, which also permitted evaluation of PCR efficiencies using the formula E=10^{[-1/slope]}. The efficiency of amplification was checked for all targets by performing a series of serial dilutions of the template for each primer pair in triplicate. Real-time PCR data quality control calculations were determined by the SAS program as described previously [101] and the calculated PCR efficiency was between 95%-99%. Quantitative RT-PCR was conducted for different cell-line derived RNAs, and the specificity of each amplified product was confirmed by melting curve analysis. Results were expressed as copy number of specific isoforms per 100 ng of total RNA.

Extraction of RNA from Subcellular Fractions and Validation

Nucleoli were purified from a confluent monolayer of EA.hy926 cells using a modified version [102] of the original method described by Busch et al. [103]. Briefly, cells were incubated in a hypotonic solution containing 10 mM HEPES (pH 7.9), 1.5 mM MgCl₂, 2.5 mM KCl, and 0.5 mM DTT for 5 min, and then scraped and homogenized using a pre-chilled dounce homogenizer to break the cells and to release the nucleus. The homogenized subcellular fractions were centrifuged at 300 g for 5 min and the supernatant was collected as a cytosolic fraction. The nuclear pellet was resuspended in solution S1 containing 0.25 M sucrose/10 mM MgCl₂ and slowly layered onto solution S2 containing 0.35 M sucrose/10 mM MgCl₂. After centrifugation at 1500 g for 5 min, the nuclear pellet was resuspended in solution S2 and subsequently sonicated to completely disrupt the nuclear membrane. The sub-nuclear fractions were then layered on top of solution S3 containing 0.88 M sucrose/10 mM MgCl₂ and centrifuged at 2800 g for 5 min to obtain the nucleolar pellet. The supernatant was collected as the nucleoplasm fraction (nucleus-nucleolus). The nucleolar pellet was washed once with solution S2 and frozen immediately. For immunofluorescence-based evaluation, the purified nucleoli were immobilized on poly-L-lysine (Sigma) coated slides and air-dried. After rehydration with phosphate-buffered saline (PBS: 137 mM NaCl, 2.7 mM KCl and 10 mM phosphate buffer solution, pH 7.4) and a brief fixation (10 min) with freshly prepared 2% paraformaldehyde in PBS, the nucleoli were incubated with monoclonal anti-NCL/NLM1 antibody for 30 min, then washed and incubated with FITC conjugated secondary antibodies for 30 min. The slides were washed with PBS and counterstained with an anti-fade reagent containing DAPI. Images were captured using an Axiovision 4.1 Zeiss microscope.

Genomic DNA-free nuclear and nucleolar RNA were extracted from the harvested pellets using the Monarch Total RNA miniprep kit (catalogue number: T2010G, New England Biolabs). The cytosol and nucleoplasm fractions were in a large volume and the total RNAs from these fractions were harvested by the method of Max et al. [104]. Briefly, the cytosol/nucleoplasm fractions were mixed with an equal volume of a buffer containing 4 M citric acid, 4 M NaOH, 0.8% (w/v) sarkosyl, 3 M guanidium thiocyanate, and 80% (v/v) 0.1 M citrate buffer (pH 4.3)-saturated Phenol solution. The solution was briefly vortexed and mixed with 100 μL/mL chloroform and incubated at room temperature for 5 min. Subsequently, the suspension was centrifuged at 5000 g for 10 min and the supernatant was collected. RNA from the supernatant was harvested by isopropanol-mediated precipitation followed by washing with 70% ethanol. The RNA pellet was dissolved in RNase free water and stored.

### Plasmids, Cloning, and Sanger Sequencing, and Transfection

The full-length ALOX12-AS1-201 and ALOX12-AS1-202 transcripts (Transcript ID: ENST00000399540.2 and ENST00000399541.6, respectively) were cloned into the BamH1/EcoRI restriction sites of the pcDNA3.1(+) vector. During cloning, T7 and Sp6 promoter sequences were inserted into the 5′ and 3′ end of the lncRNA transcripts for in vitro transcription. The plasmids were used for the generation of FISH probes by in vitro transcription using T7/Sp6 polymerases. The ALOX12-AS1201/202 lncRNA-specific RT-PCR products were agarose gel-purified and cloned into a pCR2.1-TOPO TA Vector using the TOPO-TA cloning kit (Catalog number: 450641; ThermoFisher Scientific, Canada) as per the manufacturer's protocol. Sanger sequencing was performed at the sequencing facility of the Hospital for Sick Children, Toronto, Canada. Transfection of mRNAs was achieved using Lipofectamine® MessengerMAX™ reagent (Catalog number: LMRNA001; ThermoFisher Scientific).

### Northern Blotting

Northern blotting was performed using the NorthernMax Kit (Catalog number: AM1940, ThermoFisher Scientific) as per the manufacturer's instructions. Briefly, total RNA (20 μg) was separated using a 1% agarose gel and transferred to BrightStar™-Plus Positively Charged Nylon membranes (Catalog number: AM10104; ThermoFisher Scientific) using a downward transfer assembly. A denatured single-strand RNA ladder (Catalog number: N0362S, New England Biolabs) was used as a reference for RNA size. The transferred RNA was crosslinked by exposure to 254 nm UV light for 1 min using a UV crosslinker (model FV-UVXL1000; Fisher Scientific). Fluorescence-conjugated ALOX12-AS1 isoform-specific RNA Northern blotting probes (Table 1) were synthesized using the FISH Tag™ RNA Multicolor Kit (Catalog number: F32956; ThermoFisher Scientific). The labeled probes were hybridized for 4 hours at 60° C. in a rotating hybridization oven and subsequently visualized and imaged using the ChemiDoc MP imaging System (Bio-Rad).

### RNA-FISH

For RNA-FISH, cells were grown on coverslips and human tissue cryosections were adhered to glass slides. Human PBMs were harvested from whole blood. The harvested PBMs were suspended in RPMI media (Catalog number: 61870127; ThermoFisher Scientific) supplemented with 10% FBS and plated onto a Matrigel growth factor reduced (GFR) basement membrane matrix (catalogue number: 356238; Corning) coated glass coverslip which had been placed at the bottom of a 6 well culture dish. The plate containing the cells was centrifuged at 600 g for 10 min and incubated in a CO₂ incubator for 2 hours. The cells were then fixed and analyzed by RNA-FISH. THP-1 cells were prepared in a similar way for RNA-FISH. The cells/cryosections were fixed in 4% freshly prepared paraformaldehyde (PFA) in PBS for 20 min, followed by sequential incubations in absolute ethanol (2×5 min), xylene (30 min), absolute ethanol (2×5 min), methanol (2×5 min), 50% methanol+2% PFA+PBS+0.1% Tween-20 (5 min), 2% PFA/PBS+0.1% Tween-20 (2×10 min), 2×5 min in PBT (PBS+0.1% Tween-20), PBT+20 μg/mL proteinase K (10 min), PBT (2×5 min), 2% PFA+PBT (2×10 min), PBT (4×5 min), PBT+50% hybridization buffer (10 min), and hybridization buffer (1×30 min at 55° C.). The hybridization buffer was prepared with 50% formamide, 5×SSC, 100 μg/mL fragmented salmon sperm DNA, 50 μg/mL heparin, and 0.1% Tween-20. The labeled FISH probes were prepared as mentioned in the Northern blotting probe preparation method. In addition, fluorescein-12-UTP (Catalog number: 11427857910; Roche). was used to label probes generated by Sp6 polymerase mediated in vitro transcription. The RNA FISH probe was used as both non-fragmented and fragmented. The FISH probe was fragmented by mixing (1:1) fluorescent-labeled RNA with 2× carbonate buffer (127 mg Na₂CO₃+67.2 mg NaHCO₃, pH 10.2 with NaOH) followed by incubation at 42° C. for 30 min and subsequent addition of an equal volume of stop solution (164.1 mg sodium acetate, pH 6 with acetic acid). The fragmented as well as non-fragmented labeled FISH probes were precipitated with ethanol and dissolved in RNase free water and subsequently mixed with hybridization buffer and incubated overnight with the cells/tissues at 50° C. Following hybridization, the cells/tissues were stained with Hoechst in PBST and mounted using SlowFade™ Gold Antifade Mountant (Catalog number: 536936; ThermoFisher Technologies) and visualized using a Zeiss LSM410 confocal microscope and a Nikon Eclipse Ti2 inverted microscope. The sense templates of the ALOX12-AS1 lncRNA were used as a control for the RNA-FISH and no specific signal was detected (data not shown).

### In Vitro-Transcription, 3′-Biotinylation and Streptavidin Conjugation

The antisense (−) and sense (+) transcripts of ALOX12-AS1 isoforms were transcribed in vitro using the HiScribe™ T7/Sp6 High Yield RNA Synthesis Kit (Catalog number: E2040S & E2070S; New England Biolabs) and BamH1/EcoRI digested linearized plasmid DNA as templates, respectively. The sense strand of the antisense lncRNAs was used as a control for transfection as well as RNA-FISH experiments. The in vitro transcribed lncRNAs were 3′ end labelled with a 3′Biotin-dA(10)-5′-PO⁴ oligonucleotide RNA probe using T4 RNA Ligase 1 (ssRNA Ligase, catalog number: M0204S, NEB). A 20 μl reaction was set up at 16° C. for 16 hours using 1×T4 RNA ligase reaction buffer (50 mM Tris-HCl, pH 7.5, 10 mM MgCl2, 1 mM DTT), 15% (w/v) polyethylene glycol (PEG) 8000, 0.5 μl RNase Inhibitor, 1 μl (10 units) T4 RNA ligase 1, 1 mM ATP, 20 pmol RNA, 100 pmol RNA oligo. The reaction was stopped by column clean up with Monarch RNA clean up kit (catalog number T2040S; NEB) that filtered the excess unincorporated biotin-dAs. The purified A-tailed 3′ Biotin labeled lncRNAs were transfected into the cells using Lipofectamine® MessengerMAX™ reagent (Catalog number: LMRNA001; ThermoFisher Scientific).

Formaldehyde Crosslinked lncRNA Purification Using Streptavidin C1 Beads

The biotin-dA labelled ALOX12-AS1-201 lncRNA was transfected into HEK293 cells. The transfected cells were crosslinked for 10 mins using 4% freshly prepared paraformaldehyde in PBS. The crosslinking was stopped by addition of 50 mM Tris-HCl pH 7.5 and the cells were lysed in a buffer containing 50 mM Tris-HCl pH7, 75 mM NaCl, 10 mM EDTA, 1% SDS, 1× protease inhibitor cocktail, and 30 units/μl RNAse inhibitor (catalog no:R1158, Sigma). The cell lysate was subsequently sonicated. The MyOne Streptavidin C1 (catalog number: 65001, Invitrogen) beads were resuspended 3-4 times in a binding and washing buffer containing 10 mM Tris-HCl pH 7.5, 1 mM EDTA and 2 M NaCl and then incubated with the cell lysate for 30 min at room temperature with gentle rotation of the tube. The beads were then washed 3-4 times in washing buffer. During the final wash cycle, the resuspended beads were separated into two aliquots and subsequently used for RNA and protein isolation. The RNA was isolated by resuspending the beads in a buffer (100 μl) containing 100 mM NaCl, 10 mM Tris-HCl pH 7.5, 1 mM EDTA, 0.5% SDS and treating with 5 μl proteinase K (10 mg/mL). Incubation was performed at 50° C. for 1 hour with end to end shaking in a hybridization oven. Subsequently, the RNA was eluted using Monarch RNA clean up kit (catalog number T2040S; NEB). The eluted RNA was used for cDNA preparation and subsequent RT-PCR applications. The ALOX12-AS1-201 crosslinked proteins were eluted as described previously [105].

### RNase Protection Assay

The in vitro transcribed and purified ALOX12 mRNA and ALOX12-AS1-201/202 lncRNAs were denatured and mixed in an equimolar ratio in a duplex formation buffer containing 50% formamide, 10 mM Tris-HCl pH 7.5, 300 mM NaCl and 5 mM EDTA pH 7.5, followed by overnight incubation at 37° C. in a rotating shaker. The mixture was subsequently incubated with RNaseA/T1 (1 unit/μg RNA; catalogue number: EN0551, ThermoFisher Technologies) for 2 hours and the products were separated in a denaturing 1% agarose gel and visualized.

### Western Blotting and Quantification

Relative quantification of proteins was performed as described previously [35, 106]. Table 1 lists the primary antibodies used in this study. The cell lysates were prepared in 1×RIPA lysis and extraction buffer (catalogue number: 89900, ThermoFisher Scientific) supplemented with 1× Halt protease and phosphatase inhibitor cocktail (catalogue number: 78441, ThermoFisher Scientific). The protein lysates were denatured in Laemmli buffer containing 2% SDS, 10% glycerol, 0.002% bromophenol blue, 0.75 M Tris-HCl pH 6.8, and 100 mM DTT. The immunoblots were imaged using a ChemiDoc XRS+ system (Bio-Rad) and band intensities were quantified using ImageJ (version 1.48) Software [107]. Due to the possibility of inconsistency in the relative expression of housekeeping genes, we normalized all proteins of interest based on the intensity of the Oriole-stained protein profile [92].

### Immunofluorescence

The THP-1 cells were differentiated on glass cover slips for immunofluorescence-base studies. The THP-1 cell suspension was briefly centrifuged at 600 g for 5 min on a coverslip in a 6 well plate. The cells were fixed for 10 min in 4% paraformaldehyde, permeabilized using 0.25% Triton X-100 in 1×PBS, blocked (1% BSA), and immunostained using appropriate primary antibodies and fluorescent-conjugated secondary antibodies. Finally, the cells were washed, counter-stained (Hoechst), mounted in SlowFade™ Gold Antifade mounting media (Thermo Fisher Scientific, S36936) and visualized.

### Measurement of the Catalytic Activity of ALOX12.

ALOX12 catalytic activity was measured in a cell-free system using cell lysates derived from unactivated human platelets (positive control) and Phorbol ester (12-O-Tetradecanoylphorbol 13-acetate: TPA)/vehicle (DMSO/ethanol)-treated THP-1. ALOX12 converts arachidonate to the bioactive lipid 12(S)-hydroperoxyeicosatetraenoate (12S-HpETE) [59]. The 12S-HpETE is subsequently reduced by cellular peroxidases to 12(S)-hydroxyeicosatetraenoic acid (12S-HETE) [59]. The cell lysates prepared in 1×PBS supplemented with 0.5% NP40, 1× Halt protease and phosphatase inhibitor cocktail 1 were incubated with 50 μM arachidonic acid (catalogue number: A0781, Tokyo Chemical Industry, USA) for 15 min at 37° C. and the amount of 12S-HETE was quantitatively measured using a commercially available Enzyme-Linked ImmunoAssay (ELISA) kit (catalogue number: ADI-900-050, Enzo Life Sciences Inc., USA) as per the manufacturer's instructions [108]. Freshly harvested unactivated platelet-derived protein lysates served a positive control for ALOX12 enzyme activity [59]. The lysates were prepared from freshly harvested platelets obtained from consented healthy human donors as part of an ongoing study which is approved by the Biomedical Research Ethics Board of the University of Manitoba. The platelets were resuspended and lysed in Tyrode's buffer (134 mM NaCl, 12 mM NaHCO₃, 2.9 mM KCl, 0.34 mM Na₂HPO₄, 1 mM MgCl₂, 10 mM HEPES, pH 7.4), with freshly added BSA (3 mg/ml). The platelet lysate reaction product was diluted (1:16) to fit within the range of the assay.

### Statistical Analysis

Statistical analysis was performed using Prism version 7.00 (GraphPad Software). The means of more than 2 groups were compared using one-way ANOVA (randomized) [109, 110], followed by Dunnett's post hoc multiple comparison test to compare the means of multiple experimental groups against a control group mean [109]. Comparisons between two groups were performed using Student's t-test (unpaired). Differences were considered significant at P<0.05.

While the preferred embodiments of the invention have been described above, it will be recognized and understood that various modifications may be made therein, and the appended claims are intended to cover all such modifications which may fall within the spirit and scope of the invention.

### REFERENCES

1. Hamberg, M. and B. Samuelsson, Prostaglandin endoperoxides. Novel transformations of arachidonic acid in human platelets. Proc Natl Acad Sci USA, 1974. 71(9): p. 3400-4.
2. Nugteren, D. H., Arachidonate lipoxygenase in blood platelets. Biochim Biophys Acta, 1975. 380(2): p. 299-307.
3. Hussain, H., et al., Epidermis contains platelet-type 12-lipoxygenase that is overexpressed in germinal layer keratinocytes in psoriasis. Am J Physiol, 1994. 266(1 Pt 1): p. C243-53.
4. Zhao, H., et al., Lipoxygenase mRNA in cultured human epidermal and oral keratinocytes. J Lipid Res, 1995. 36(11): p. 2444-9.
5. Krieg, P., et al., 12-Lipoxygenase isoenzymes in mouse skin tumor development. Mol Carcinog, 1995. 14(2): p. 118-29.
6. Setty, B. N., et al., Eicosanoids in sickle cell disease: potential relevance of 12(S)-hydroxy-5,8,10,14-eicosatetraenoic acid to the pathophysiology of vaso-occlusion. J Lab Clin Med, 1998. 131(4): p. 344-53.
7. Du, Y., et al., Role of oxylipins generated from dietary PUFAs in the modulation of endothelial cell function. Prostaglandins Leukot Essent Fatty Acids, 2020. 160: p. 102160.
8. Scientific Opinion on the Tolerable Upper Intake Level of eicosapentaenoic acid (EPA), docosahexaenoic acid (DHA) and docosapentaenoic acid (DPA). EFSA Journal, 2012. 10(7).
9. Zheng, Z., et al., The biological role of arachidonic acid 12-lipoxygenase (ALOX12) in various human diseases. Biomed Pharmacother, 2020. 129: p. 110354.
10. Singh, N. K. and G. N. Rao, Emerging role of 12/15-Lipoxygenase (ALOX15) in human pathologies. Prog Lipid Res, 2019. 73: p. 28-45.
11. Poch, E., Role of lipoxygenase metabolites in cardiovascular disease. Curr Hypertens Rep, 2003. 5(1): p. 1-2.
12. Iversen, L. and K. Kragballe, Arachidonic acid metabolism in skin health and disease. Prostaglandins Other Lipid Mediat, 2000. 63(1-2): p. 25-42.
13. Martinez-Clemente, M., et al., Disruption of the 12/15-lipoxygenase gene (Alox15) protects hyperlipidemic mice from nonalcoholic fatty liver disease. Hepatology, 2010. 52(6): p. 1980-91.
14. Samala, N., et al., Molecular mechanisms of nonalcoholic fatty liver disease: Potential role for 12-lipoxygenase. J Diabetes Complications, 2017. 31(11): p. 1630-1637.
15. Gad, H. I., Effects of pravastatin or 12/15 lipoxygenase pathway inhibitors on indices of diabetic nephropathy in an experimental model of diabetic renal disease. Saudi Med J, 2012. 33(6): p. 608-16.
16. Klampfl, T., et al., Up-regulation of 12(S)-lipoxygenase induces a migratory phenotype in colorectal cancer cells. Exp Cell Res, 2012. 318(6): p. 768-78.
17. Chang, J., et al., 12/15 Lipoxygenase regulation of colorectal tumorigenesis is determined by the relative tumor levels of its metabolite 12-HETE and 13-HODE in animal models. Oncotarget, 2015. 6(5): p. 2879-88.
18. Kamitani, H., M. Geller, and T. Eling, The possible involvement of 15-lipoxygenase/leukocyte type 12-lipoxygenase in colorectal carcinogenesis. Adv Exp Med Biol, 1999. 469: p. 593-8.
19. J., R., et al., Role of Lipoxygenases in Pathogenesis of Cancer, in Lipoxygenases in Inflammation. 2016, Springer, Cham. p. 131-157.
20. Pratico, D., et al., 12/15-lipoxygenase is increased in Alzheimer's disease: possible involvement in brain oxidative stress. Am J Pathol, 2004. 164(5): p. 1655-62.
21. Kim, S., et al., p63 directly induces expression of Alox12, a regulator of epidermal barrierformation. Exp Dermatol, 2009. 18(12): p. 1016-21.
22. Kaur, G., et al., RUNX1/core binding factor A2 regulates platelet 12-lipoxygenase gene (ALOX12): studies in human RUNX1 haplodeficiency. Blood, 2010. 115(15): p. 3128-35.
23. Zerbino, D. R., et al., Ensembl 2018. Nucleic Acids Res, 2018. 46(D1): p. D754-D761.
24. Kuhn, H., et al., The evolutionary hypothesis of reaction specificity of mammalian ALOX15 orthologs. Prog Lipid Res, 2018. 72: p. 55-74.
25. Kent, W. J., et al., The human genome browser at UCSC. Genome Res, 2002. 12(6): p. 996-1006.
26. Ota, T., et al., Complete sequencing and characterization of 21,243 full-length human cDNAs. Nat Genet, 2004. 36(1): p. 40-5.
27. Yudate, H. T., et al., HUNT: launch of a full-length cDNA database from the Helix Research Institute. Nucleic Acids Res, 2001. 29(1): p. 185-8.
28. Uhl\tilde{e}n, M., et al., Proteomics. Tissue-based map of the human proteome. Science, 2015. 347(6220): p. 1260419.
29. Pelechano, V. and L. M. Steinmetz, Gene regulation by antisense transcription. Nat Rev Genet, 2013. 14(12): p. 880-93.
30. Latos, P. A., et al., Airn transcriptional overlap, but not its lncRNA products, induces imprinted Igf2r silencing. Science, 2012. 338(6113): p. 1469-72.
31. Fernandes, J. C. R., et al., Long Non-Coding RNAs in the Regulation of Gene Expression: Physiology and Disease. Noncoding RNA, 2019. 5(1).
32. Lorenz, R., et al., ViennaRNA Package 2.0. Algorithms for Molecular Biology, 2011. 6(1): p. 26.
33. Pauls, S. D., et al., Impact of Age, Menopause, and Obesity on Oxylipins Linked to Vascular Health. Arterioscler Thromb Vasc Biol, 2020: p. Atvbaha120315133.
34. Kuhn, H., S. Banthiya, and K. van Leyen, Mammalian lipoxygenases and their biological relevance. Biochim Biophys Acta, 2015. 1851(4): p. 308-30.
35. Sabbir, M. G., C. G. Taylor, and P. Zahradka, Hypomorphic CAMKK2 in EA.hy926 endothelial cells causes abnormal transferrin trafficking, iron homeostasis and glucose metabolism. Biochim Biophys Acta Mol Cell Res, 2020: p. 118763.
36. Dempsey, E. C., et al., Protein kinase C isozymes and the regulation of diverse cell responses. Am J Physiol Lung Cell Mol Physiol, 2000. 279(3): p. L429-38.
37. Grammatikakis, I., K. Abdelmohsen, and M. Gorospe, Posttranslational control of HuR function. Wiley Interdiscip Rev RNA, 2017. 8(1).
38. Doller, A., et al., Protein kinase C alpha-dependent phosphorylation of the mRNA-stabilizing factor HuR: implications for posttranscriptional regulation of cyclooxygenase-2. Mol Biol Cell, 2007. 18(6): p. 2137-48.
39. Yoon, J. H., et al., Scaffold function of long non-coding RNA HOTAIR in protein ubiquitination. Nat Commun, 2013. 4: p. 2939.
40. Yoon, J. H., et al., LincRNA-p21 suppresses target mRNA translation. Mol Cell, 2012. 47(4): p. 648-55.
41. Gerstmeier, J., et al., An experimental cell-based model for studying the cell biology and molecular pharmacology of 5-lipoxygenase-activating protein in leukotriene biosynthesis. Biochim Biophys Acta, 2014. 1840(9): p. 2961-9.
42. Liening, S., et al., 12-Oxo-10-glutathionyl-5,8,14-eicosatrienoic acid (TOG10), a novel glutathione-containing eicosanoid generated via the 12-lipoxygenase pathway in human platelets. Prostaglandins Other Lipid Mediat, 2021. 152: p. 106480.
43. Dilly, A.-K., et al., Convergence of eicosanoid and integrin biology: Role of Src in 12-LOX activation. Experimental cell research, 2017. 351(1): p. 1-10.
44. Shah, R., M. S. Shchepinov, and D. A. Pratt, Resolving the Role of Lipoxygenases in the Initiation and Execution of Ferroptosis. ACS central science, 2018. 4(3): p. 387-396.
45. Gregus, A. M., et al., Systematic analysis of rat 12/15-lipoxygenase enzymes reveals critical role for spinal eLOX3 hepoxilin synthase activity in inflammatory hyperalgesia. FASEB J, 2013. 27(5): p. 1939-49.
46. Ponicsan, S. L., J. F. Kugel, and J. A. Goodrich, Genomic gems: SINE RNAs regulate mRNA production. Curr Opin Genet Dev, 2010. 20(2): p. 149-55.
47. Carrieri, C., et al., Long non-coding antisense RNA controls Uchl1 translation through an embedded SINEB2 repeat. Nature, 2012. 491(7424): p. 454-7.
48. Huarte, M., et al., A large intergenic noncoding RNA induced by p53 mediates global gene repression in the p53 response. Cell, 2010. 142(3): p. 409-19.
49. Cabili, M. N., et al., Localization and abundance analysis of human lncRNAs at single-cell and single-molecule resolution. Genome Biol, 2015. 16: p. 20.
50. Djebali, S., et al., Landscape of transcription in human cells. Nature, 2012. 489(7414): p. 101-8.
51. Kojima, K. K., Human transposable elements in Repbase: genomic footprints from fish to humans. Mobile DNA, 2018. 9(1): p. 2.
52. Akopian, D., et al., Signal recognition particle: an essential protein-targeting machine. Annu Rev Biochem, 2013. 82: p. 693-721.
53. Zwieb, C., et al., A nomenclature for all signal recognition particle RNAs. RNA, 2005. 11(1): p. 7-13.
54. Consortium, G. T., The Genotype-Tissue Expression (GTEx) project. Nat Genet, 2013. 45(6): p. 580-5.
55. Jarroux, J., A. Morillon, and M. Pinskaya, History, Discovery, and Classification of lncRNAs. Adv Exp Med Biol, 2017. 1008: p. 1-46.
56. Jiang, C., et al., Identifying and functionally characterizing tissue-specific and ubiquitously expressed human lncRNAs. Oncotarget, 2016. 7(6): p. 7120-33.
57. Zhang, B., et al., A novel RNA motif mediates the strict nuclear localization of a long noncoding RNA. Mol Cell Biol, 2014. 34(12): p. 2318-29.
58. Chen, L. L., Linking Long Noncoding RNA Localization and Function. Trends Biochem Sci, 2016. 41(9): p. 761-772.
59. Ikei, K. N., et al., Investigations of human platelet-type 12-lipoxygenase: role of lipoxygenase products in platelet activation. J Lipid Res, 2012. 53(12): p. 2546-59.
60. Hada, T., et al., Catalytic properties of human platelet 12-lipoxygenase as compared with the enzymes of other origins. Biochim Biophys Acta, 1991. 1083(1): p. 89-93.
61. Pauls, S. D., et al., Alpha-linolenic acid enhances the phagocytic and secretory functions of alternatively activated macrophages in part via changes to the oxylipin profile. Int J Biochem Cell Biol, 2020. 119: p. 105662.
62. Voskuil, J. L., The challenges with the validation of research antibodies. F1000Res, 2017. 6: p. 161.
63. Pillai-Kastoori, L., et al., Antibody validation for Western blot: By the user, for the user. J Biol Chem, 2020. 295(4): p. 926-939.
64. Hornbeck, P. V., et al., PhosphoSitePlus, 2014: mutations, PTMs and recalibrations. Nucleic Acids Res, 2015. 43(Database issue): p. D512-20.
65. Hornbeck, P. V., et al., PhosphoSitePlus: a comprehensive resource for investigating the structure and function of experimentally determined post-translational modifications in man and mouse. Nucleic Acids Res, 2012. 40(Database issue): p.

D261-70.
66. Markoutsa, S., et al., Analysis of 5-lipoxygenase phosphorylation on molecular level by MALDI-MS. Febs j, 2014.281(8): p. 193147.
67. Hemak, J., D. Gale, and T. G. Brock, Structural characterization of the catalytic domain of the human 5-lipoxygenase enzyme. Molecular modeling annual, 2002. 8(4): p. 102-112.
68. Mazhar, S., et al., Challenges and Reinterpretation of Antibody-Based Research on Phosphorylation of Tyr(307) on PP2Ac. Cell Rep, 2020. 30(9): p. 3164-3170 e3.
69. Fulzele, A. and E. J. Bennett, Ubiquitin diGLY Proteomics as an Approach to Identify and Quantify the Ubiquitin-Modified Proteome. Methods Mol Biol, 2018. 1844: p. 363-384.
70. Bass, J. J., et al., An overview of technical considerations for Western blotting applications to physiological research. Scandinavian journal of medicine & science in sports, 2017. 27(1): p. 4-25.
71. Kim, E., S. J. Muga, and S. M. Fischer, Identification and characterization of a phorbol ester-responsive element in the murine 8S-lipoxygenase gene. J Biol Chem, 2004. 279(12): p. 11188-97.
72. de Juanes, S., et al., Development of an ichthyosiform phenotype in Alox12b-deficient mouse skin transplants. J Invest Dermatol, 2009. 129(6): p. 1429-36.
73. Liaw, Y. W., et al., Induction of 12-lipoxygenase expression by phorbol 12-myristate 13-acetate in human epidermoid carcinoma A431 cells. Biochim Biophys Acta, 1998. 1389(1): p. 23-33.
74. Liu, Y. W., et al., Induction of 12-lipoxygenase expression by epidermal growth factor is mediated by protein kinase C in A431 cells. J Pharmacol Exp Ther, 1994. 271(1): p. 567-73.
75. Horn, F., et al., Decreased protein kinase C activity in psoriatic versus normal epidermis. J Invest Dermatol, 1987. 88(2): p. 220-2.
76. Damtew, B. and P. J. Spagnuolo, Tumor cell-endothelial cell interactions: evidence for roles for lipoxygenase products of arachidonic acid in metastasis. Prostaglandins Leukot Essent Fatty Acids, 1997. 56(4): p. 295-300.
77. Damtew, B., et al., Human neutrophil adhesion to bovine aortic endothelium. Evidence for endothelial lipoxygenase activity. J Leukoc Biol, 1993. 53(6): p. 619-29.
78. Honn, K. V., et al., Lipoxygenase products regulate IRGpIIb/IIIa receptor mediated adhesion of tumor cells to endothelial cells, subendothelial matrix and fibronectin. Proc Soc Exp Biol Med, 1988. 189(1): p. 130-5.
79. Tang, D. G., et al., Transcriptional activation of endothelial cell integrin alpha v by protein kinase C activator 12(S)-HETE. J Cell Sci, 1995. 108 (Pt 7): p. 2629-44.
80. Zuberek, M., et al., Glucose availability determines silver nanoparticles toxicity in HepG2. Journal of Nanobiotechnology, 2015. 13(1): p. 72.
81. Vendel Nielsen, L., et al., Effects of elaidic acid on lipid metabolism in HepG2 cells, investigated by an integrated approach of lipidomics, transcriptomics and proteomics. PLoS One, 2013. 8(9): p. e74283.
82. Schaar, D., et al., A phase I clinical trial of 12-O-tetradecanoylphorbol-13-acetate for patients with relapsed/refractory malignancies. Cancer Chemother Pharmacol, 2006. 57(6): p. 789-95.
83. Giard, D. J., et al., In vitro cultivation of human tumors: establishment of cell lines derivedfrom a series of solid tumors. J Natl Cancer Inst, 1973. 51(5): p. 1417-23.
84. Edgell, C. J., C. C. McDonald, and J. B. Graham, Permanent cell line expressing human factor VIII-related antigen established by hybridization. Proc Natl Acad Sci USA, 1983. 80(12): p. 3734-7.
85. Tsuchiya, S., et al., Establishment and characterization of a human acute monocytic leukemia cell line (THP-1). Int J Cancer, 1980. 26(2): p. 171-6.
86. Shaw, G., et al., Preferential transformation of human neuronal cells by human adenoviruses and the origin of HEK 293 cells. FASEB J, 2002. 16(8): p. 869-71.
87. Darlington, G. J., J. H. Kelly, and G. J. Buffone, Growth and hepatospecific gene expression of human hepatoma cells in a defined medium. In Vitro Cell Dev Biol, 1987. 23(5): p. 349-54.
88. Wilkins, M. R., et al., Protein identification and analysis tools in the ExPASy server. Methods Mol Biol, 1999. 112: p. 531-52.
89. Wuest, S. J., et al., Expression and regulation of 12/15-lipoxygenases in human primary macrophages. Atherosclerosis, 2012. 225(1): p. 121-7.
90. Poirier, S. J., et al., LPS induces ALOX5 promoter activation and 5-lipoxygenase expression in human monocytic cells. Prostaglandins Leukot Essent Fatty Acids, 2020. 154: p. 102078.
91. Pauls, S. D., et al., Anti-inflammatory effects of α-linolenic acid in M1-like macrophages are associated with enhanced production of oxylipins from α-linolenic and linoleic acid. J Nutr Biochem, 2018. 57: p. 121-129.
92. Sabbir, M. G., C. G. Taylor, and P. Zahradka, Hypomorphic CAMKK2 in EA.hy926 endothelial cells causes abnormal transferrin trafficking, iron homeostasis and glucose metabolism. Biochim Biophys Acta Mol Cell Res, 2020. 1867(10): p. 118763.
93. Schmid, M. C., et al., Integrin CD11b activation drives anti-tumor innate immunity. Nature Communications, 2018. 9(1): p. 5379.
94. Rosetti, F. and T. N. Mayadas, The many faces of Mac-1 in autoimmune disease. Immunol Rev, 2016. 269(1): p. 175-93.
95. Chistiakov, D. A., et al., CD68/macrosialin: not just a histochemical marker. Laboratory Investigation, 2017. 97(1): p. 4-13.
96. Hamberg, M. and B. Samuelsson, Prostaglandin endoperoxides. Novel transformations of arachidonic acid in human platelets. Proceedings of the National Academy of Sciences of the United States of America, 1974. 71(9): p. 3400-3404.
97. Chen, Y. Q., et al., Endogenous 12(S)-HETE production by tumor cells and its role in metastasis. Cancer Res, 1994. 54(6): p. 1574-9.
98. Yoon, J. H., K. Abdelmohsen, and M. Gorospe, Posttranscriptional gene regulation by long noncoding RNA. J Mol Biol, 2013. 425(19): p. 3723-30.
99. Carey, M. F., C. L. Peterson, and S. T. Smale, The RNase protection assay. Cold Spring Harb Protoc, 2013. 2013(3).
100. Fernandez, F. J., et al., Protein-tRNA Agarose Gel Retardation Assays for the Analysis of the N 6-threonylcarbamoyladenosine TcdA Function. Journal of visualized experiments: JoVE, 2017(124): p. 55638.
101. Sabbir, M. G., et al., Identification and characterization of Dlc1 isoforms in the mouse and study of the biological function of a single gene trapped isoform. BMC Biol, 2010. 8: p. 17.
102. Andersen, J. S., et al., Directed proteomic analysis of the human nucleolus. Curr Biol, 2002. 12(1): p. 1-11.
103. Busch, H., et al., Isolation of Nucleoli. Exp Cell Res, 1963. 24: p. SUPPL9:150-63.
104. Max, K. E. A., et al., Human plasma and serum extracellular small RNA reference profiles and their clinical utility. Proc Natl Acad Sci USA, 2018. 115(23): p. E5334-E5343.
105. Chu, C., et al., Systematic discovery of Xist RNA binding proteins. Cell, 2015. 161(2): p. 404-16.
106. Sabbir, M. G., CAMKK2-CAMK4 signaling regulates transferrin trafficking, turnover, and iron homeostasis. Cell Commun Signal, 2020. 18(1): p. 80.
107. Hartig, S. M., Basic image analysis and manipulation in ImageJ. Curr Protoc Mol Biol, 2013. Chapter 14: p. Unit14 15.
108. Olechowski, B., et al., Changes in platelet function with inflammation in patients undergoing vascular surgery. Platelets, 2019. 30(2): p. 190-198.
109. Dunn, O. J., Multiple Comparisons Using Rank Sums. Technometrics, 1964. 6(3): p. 241-252.
110. Siegel, S., ed. Nonparametric statistics for the behavioral sciences. McGraw-Hill series in psychology. 1956, McGraw-Hill: New York. pages 312.
111. Huang, Y., et al., Biological properties of baicalein in cardiovascular system. Curr Drug Targets Cardiovasc Haematol Disord, 2005. 5(2): p. 177-84.
112. Shen, Y. C., et al., Mechanisms in mediating the anti-inflammatory effects of baicalin and baicalein in human leukocytes. Eur J Pharmacol, 2003. 465(1-2): p. 171-81.
113. Butenko, I. G., S. V. Gladtchenko, and S. V. Galushko, Anti-inflammatory properties and inhibition of leukotriene C4 biosynthesis in vitro by flavonoid baicalein from Scutellaria baicalensis georgy roots. Agents Actions, 1993. 39 Spec No: p. C49-51.
114. Deschamps, J. D., V. A. Kenyon, and T. R. Holman, Baicalein is a potent in vitro inhibitor against both reticulocyte 15-human and platelet 12-human lipoxygenases. Bioorg Med Chem, 2006. 14(12): p. 4295-301.

TABLE 1

| Gene of | | | Amplicon |
|---|---|---|---|
| interest/ | Forward primer | Reverse primer | length |
| Isoforms | (5′-3′) | (5′-3′) | (base pairs) |
| ALOX12-AS1 201 | GGGCTCTTAGTATCGGA | GCCCAACAGTAAGGTGGAT | 494 |
| | GGATTG (SEQ ID NO: 12) | AGA (SEQ ID NO: 13) | |
| ALOX12-AS1-202 | GCTCTTAGTATCGGAGG | CAGAGCTCAGAAAGGAGAT | 496 |
| | ATTGGA (SEQ ID NO: 14) | GAAAC SEQ ID NO: 15) | |
| ALOX12-AS1-203 | AAAGTCTAACTGGCGG | CTTATGCTAAGGTTTCAGG | 420 |
| | AACTC (SEQ ID NO: 16) | TGTATTG (SEQ ID NO: 17) | |
| ALOX12-AS1-204 | ACCTGAAATTCCTTAGT | AGTTCCTCAATGGTGCCAA | 308 |
| | CTCTCCT (SEQ ID NO: 18) | C (SEQ ID NO: 19) | |
| ALOX12-AS1-205 | AAAGTCTAACTGGCGG | CTTATGCTAAGGTTTCAGG | 360 |
| | AACTC (SEQ ID NO: 20) | TGTATTG (SEQ ID NO: 21) | |
| ALOX12-AS1-206 | GGGCTCTTAGTATCGGA | TGAAGGGAGGAAATGGTA | 400 |
| | GGATTG (SEQ ID NO: 22) | GCTG (SEQ ID NO: 23) | |
| ALOX12-AS1-207 | TCCTTAGTCTCTCCTGT | GTTCAGCTACCAGTTCCTC | 481 |
| | GTTGG (SEQ ID NO: 24) | AATG (SEQ ID NO: 25) | |
| ALOX12-AS1-208 | ACCTGAAATTCCTTAGT | ACTGTTCTCTCCTTAGTAGT | 402 |
| | CTCTCCT (SEQ ID NO: 26) | GTGG (SEQ ID NO: 27) | |
| ALOX12-AS1-209 | GCTCTTAGTATCGGAGG | GTGTATTGATGGTATCCAG | 390 |
| | ATTGGA (SEQ ID NO: 28) | ATCAGC (SEQ ID NO: 29) | |
| GAPDH | ACGGGAAGCTTGTCATC | CCAGTAGAGGCAGGGATGA | 445 |
| | AATGG (SEQ ID NO: 30) | TGT (SEQ ID NO: 31) | |
| ALOXE3 | GACGTCAACAGCTTTCA | AGATCGTCTTGGTGGCTGA | 184 |
| | GGAGATG (SEQ ID | GTAT (SEQ ID NO: 33) | |
| | NO: 32) | | |
| ALOX5 | GGCAGGAAGACCTGAT | GCAGCTCAAAGTCCACGAT | 189 |
| | GTTTGG (SEQ ID NO: 34) | GAA (SEQ ID NO: 35) | |
| ALOX12 | GAGAAGAGGCTGGACT | CCATTGAGGAACTGGTAGC | 200 |
| | TTGAATGG (SEQ ID | TGAAC (SEQ ID NO: 37) | |
| | NO: 36) | | |
| RNASEK | GCTTTCTCCCACCGCTT | CCGAACTTGGCAGAAAGAG | 324 |
| | TCG (SEQ ID NO: 38) | AAG (SEQ ID NO: 39) | |

Sanger Sequencing primers

| | |
|---|---|
| ALOX12F1 | TACCGCATCCGCGTG |
| | (SEQ ID NO: 40) |
| ALOX12F2 | TACAACCGCGTGCAG |
| | (SEQ ID NO: 41) |
| ALOX12F3 | CAGGATGATGAGTTGTT |
| | C (SEQ ID NO: 42) |
| ALOX12F4 | CTCGTTATGCTGAAGAT |
| | G (SEQ ID NO: 43) |
| ALOX12F5 | TCTTCTCCGGGTCGTA |
| | (SEQ ID NO: 44) |
| ALOX12R1 | CTGTTCTCTATGCAGCT |
| | (SEQ ID NO: 45) |
| ALOX12R2 | CTGAAGTTCTTTCTCCA |
| | G (SEQ ID NO: 46) |
| ALOX12R3 | CATCTTCAGCATAACGA |
| | G (SEQ ID NO: 47) |

RNA FISH probe sequence (5′-3′)

| | |
|---|---|
| ALOX12-AS1- | TGAACTCCCATTCCGTATATGACATTATAGTCAGCATTCTTGGGGAAA |
| 201 | CAGAGATGACTAAGTAAGATTATTGCCTTCTAGGAGGTTATAGTCTA |
| | GCAGGAGGACTGTGAACCCTCCCAGCCTGCCGCTCGCTAGCTGGAGT |
| | GCCCTGATGTCAGGTGGAAGGCAGGAGGGACTGGAAGAAGGGGGTC |
| | CAGAAGGAGGCCCTAACAGTGGTGTGGGAGAAACCTGCTGCTTTGGG |
| | GTCGGCGCTCAGATGGTGGTGGAATGTGAAGGAGAAGTAGGAGGTGT |
| | AAGATTATTGACAGTTCTTGTCACCTGACTGTTAAGAATAGTGAGAG |
| | AAGGCCAGGTGCAGTGGCTCACACCTGTAATCCCAGCACTTTGGGAG |
| | GCCGAGGCAGGCAGATTACTTGAGCCCAGAAGTTCAAGACCAGCCTG |
| | GGCAATATGGCGAAACCCCATCTCTAC (SEQ ID NO: 48) |
| ALOX12-AS1- | AGACAGCATCTCGTATGTTGCCCAGGCTGGCCTTGAAAGCCTGGCCTC |
| 202 | AAGCCATCTTCCTGCCTCAGCCTCCCAAGTAGCTGGGATCACAGGGTT |
| | GTGGCATCACAGCTGGCTATATTCTTAACATTATTTTGTAACCATTCC |
| | AACCCCCAGAAATTTCTCTCTGGCTGACTTGATCCACAGCGCCTCCAT |
| | CGCCATCCCTGAGTGCCTTGTTGTGGAAAATCTTACTTTATCTTGGTTC |
| | TGTTTGGTATAATCGGGGAAAGTCTGTATTCTTTCATTATGTAAAACA |
| | ACTTATCTCTCATTGTTTCATCTCCTTTCTGAGCTCTGCTCTGCCAGCT |
| | CTCTTTCCAAAACCAAAATGGCTCTTCAAGTTATTTTGTAAATAATAA |
| | TGGGCCATCTACTTCTTAACATAAATGAATGATTTTCCAAGGTCAA |
| | (SEQ ID NO: 49) |

BAC clone

| Name | Catalogue/clone number | | Source | |
|---|---|---|---|---|
| BAC clone | RP11-61B20 | | BACPAC genomics | |
| List of antibodies | | | | |
| Protein of | | | Catalogue/ | |
| interest | Source | Type | clone | Lot number |
| ALOX5 | SCBT | Mouse Monoclonal | Sc136195/33 | J2417 |
| ALOX12 | Boster | Rabbit Polyclonal | PA1485-1 | 0141412C0385103 |
| ALOX12 | Boster | Rabbit Polyclonal | A02275-1 | 0021612DA8775124 |
| ALOX12 | SCBT | Mouse Monoclonal | Sc-365194 | B0119, C2817 |
| CD68 | Abcam | Mouse monoclonal | Ab955 (KP1) | L2786 |
| Flag | Sigma | Mouse Monoclonal | F3165 | SLRW9109 |
| Histone H3 | CST | Rabbit Monoclonal | 4499S/D1H2 | 9 |
| ITGAM | Abcam | Rabbit monoclonal | Ab52478 | GR265663-2 |
| NCL/C23 | SCBT | Mouse Monoclonal | Sc-17826/D6 | G0913 |
| NPM1/B23 | SCBT | Mouse Monoclonal | Sc- | F2212 |
| | | | 53175/NA24 | |
| RPS6 | CST | Rabbit Monoclonal | 2217S/5G10 | 5 |

CST: Cell Signaling Technology;
SCBT: Santa Cruz Biotechnology

1. A method for treating a disease or disorder associated with Arachidonate 12-lipoxygenase (ALOX12) protein levels, said method comprising:
administering an effective amount of an ALOX12-AS1 modulating compound to an individual in need of such treatment, the effective amount altering ALOX12 mRNA-ALOX12-AS1 lncRNA interaction and thereby altering ALOX12 protein levels compared to an untreated control.

2. The method according to claim 1 wherein the ALOX12-AS1 modulating compound promotes nucleo-cytoplasmic translocation of ALOX12-AS1 lncRNA.

3. The method according to claim 1 wherein the ALOX12-AS1 modulating compound is a protein kinase C inhibitor.

4. The method according to claim 1 wherein the ALOX12-AS1 modulating compound is a phorbol ester.

5. The method according to claim 1 wherein the ALOX12-AS1 modulating compound is an ALOX12-AS1 lncRNA antisense oligonucleotide.

6. The method according to claim 1 wherein the disease or disorder is selected from the group consisting of: cardiovascular disease, a skin disorder, a skin inflammatory disease, non-alcoholic fatty liver disease, psoriasis, diabetic nephropathy, platelet agglutination, cancer and Alzheimer's disease.

7. A method for determining if a patient is a candidate for further screening for a disease characterized by abnormal ALOX12 protein levels comprising:
measuring expression levels of ALOX-AS1 lncRNA and a reference lncRNA in a blood sample taken from the patient;
comparing the patient ALOX12-AS1 lncRNA expression level to a control ALOX12-AS1 lncRNA expression level, thereby providing an ALOX12-AS1 lncRNA ratio;
comparing the patient reference lncRNA expression level and to a control reference lncRNA expression level, thereby providing a reference lncRNA ratio, wherein:
if the ALOX12-AS1 lncRNA ratio is greater than the reference lncRNA ratio, the individual is screen for an acute or chronic inflammatory disease;
if the ALOX12-AS1 lncRNA ratio is lower than the reference lncRNA ratio, the individual is screened for a decreased inflammatory response disease.

8. The method according to claim 7 wherein the reference lncRNA is selected from the group consisting of: RNA Component Of Signal Recognition Particle 7SL1 (RN7SL1), Metastasis Associated Lung Adenocarcinoma Transcript 1 (MALAT1), H19 Imprinted Maternally Expressed Transcript (H19), and Taurine Up-Regulated 1 (TUG1).

9. A method for determining if a compound of interest is an ALOX12 modulating compound comprising:
(a) growing a culture of cells in the presence of a compound of interest;
(b) after a suitable growth interval, measuring ALOX12 protein levels in the cells of the culture;
(c) comparing the ALOX12 protein levels to control ALOX12 protein levels from a culture of similar cells grown under similar growth conditions except for presence of the compound of interest,
wherein
(d) if ALOX12 protein levels are elevated compared to the control ALOX12 protein levels, the compound is an ALOX12 activator; or
(e) if ALOX12 protein levels are reduced compared to the control ALOX12 protein levels, the compound is an ALOX12 inhibitor.

10. The method according to claim 9 wherein the compound identified in step (d) is characterized for ALOX12-AS1 lncRNA nucleo-cytoplasm translocation activation.

11. The method according to claim 9 wherein the compound identified in step (e) is characterized for ALOX12-AS1 lncRNA nucleo-cytoplasm translocation inhibition.


SEQUENCE LISTING (49 sequences)

SEQ ID NO: 1 - DNA, 211 bp, Homo sapiens
  source 1..211: mol_type = genomic DNA
cttgaagatggggtgcagtcctgggaggcaccgcatggtggcgacagcgatgacctcagccaccaggtgagtgttccagcaagtgatactggatctcgtgcagttggaaatctgaatttcggacccaggactttgccaggagccaggcaagtggggggtctgagggcaggaacagtgttggggttggagagctggggttgggaggctgaat

SEQ ID NO: 2 - DNA, 55 bp, Homo sapiens
  source 1..55: mol_type = genomic DNA
ctggccctggttgatggcggcatgctgggcagtgcacgtgaagacgcaacatgag

SEQ ID NO: 3 - DNA, 20 bp, synthetic construct
  misc_feature 1..20: note = synthetic antisense oligonucleotide gapmer 1
  misc_feature 1..20: note = phosphorothioate bonds between each base
  modified_base 1: mod_base = OTHER; note = 2'-O-methyl-adenosine
  modified_base 2: mod_base = OTHER; note = 2'-O-methyl- guanosine
  source 1..20: mol_type = other DNA
  modified_base 3: mod_base = OTHER; note = 2'-O-methyl-uracil
  modified_base 4: mod_base = OTHER; note = 2'-O-methyl-guanosine
  modified_base 5: mod_base = OTHER; note = 2-O-methyl-adenosine
  modified_base 16: mod_base = OTHER; note = 2'-O-methyl uracil
  modified_base 17: mod_base = OTHER; note = 2'-O-methyl-guanosine
  modified_base 18: mod_base = OTHER; note = 2'-O-methyl guanosine
  modified_base 19: mod_base = OTHER; note = 2'-O-methyl uracil
  modified_base 20: mod_base = OTHER; note = 2'-O-methyl adenosine
agngaagggaggaaanggna

SEQ ID NO: 4 - DNA, 20 bp, synthetic construct
  misc_feature 1..20: note = antisense oligonucleotide gapmer 2
  misc_feature 1..20: note = phosphorothioate bonds between each base
  source 1..20: mol_type = other DNA
  modified_base 1: mod_base = OTHER; note = 2'-O-methyl guanosine
  modified_base 2: mod_base = OTHER; note = 2'-O-methyl uracil
  modified_base 3: mod_base = OTHER; note = 2'-O-methyl adenosine
  modified_base 4: mod_base = OTHER; note = 2'-O-methyl uracil
  modified_base 5: mod_base = OTHER; note = 2'-O-methyl guanosine
  modified_base 16: mod_base = OTHER; note = 2'-O-methyl adenosine
  modified_base 17: mod_base = OTHER; note = 2'-O-methyl adenosine
  modified_base 18: mod_base = OTHER; note = 2'-O-methyl uracil
  modified_base 19: mod_base = OTHER; note = 2'-O-methyl guanosine
  modified_base 20: mod_base = OTHER; note = 2'-O-methyl adenosine
gnangttagcatggtaanga

SEQ ID NO: 5 - DNA, 20 bp, synthetic construct
  misc_feature 1..20: note = Antisense oligonucleotide gapmer 3
  misc_feature 1..20: note = phosphorothioate bonds between bases
  source 1..20: mol_type = other DNA
  modified_base 1: mod_base = OTHER; note = 2'-O-methyl guanosine
  modified_base 2: mod_base = OTHER; note = 2'-O-methyl uracil
  modified_base 3: mod_base = OTHER; note = 2'-O-methyl adenosine
  modified_base 4: mod_base = OTHER; note = 2'-O-methyl cytosine
  modified_base 5: mod_base = OTHER; note = 2'-O-methyl uracil
  modified_base 16: mod_base = OTHER; note = 2'-O-methyl uracil
  modified_base 17: mod_base = OTHER; note = 2'-O-methyl uracil
  modified_base 18: mod_base = OTHER; note = 2'-O-methyl adenosine
  modified_base 19: mod_base = OTHER; note = 2'-O-methyl adenosine
  modified_base 20: mod_base = OTHER; note = 2'-O-methyl adenosine
gnacntattaatctannaaa

SEQ ID NO: 6 - DNA, 20 bp, synthetic construct
  misc_feature 1..20: note = antisense oligonucleotide gapmer 4
  misc_feature 1..20: note = phosphorothioate bonds between bases
  source 1..20: mol_type = other DNA
  modified_base 1: mod_base = OTHER; note = 2'-O-methyl adenosine
  modified_base 2: mod_base = OTHER; note = 2'-O-methyl guanosine
  modified_base 3: mod_base = OTHER; note = 2'-O-methyl adenosine
  modified_base 4: mod_base = OTHER; note = 2'-O-methyl adenosine
  modified_base 5: mod_base = OTHER; note = 2'-O-methyl cytosine
  modified_base 16: mod_base = OTHER; note = 2'-O-methyl cytosine
  modified_base 17: mod_base = OTHER; note = 2'-O-methyl uracil
  modified_base 18: mod_base = OTHER; note = 2'-O-methyl uracil
  modified_base 19: mod_base = OTHER; note = 2'-O-methyl adenosine
  modified_base 20: mod_base = OTHER; note = 2'-O-methyl cytosine
agaactgtcaataatcnnac

SEQ ID NO: 7 - DNA, 20 bp, synthetic construct
  misc_feature 1..20: note = antisense oligonucleotide gapmer 5
  misc_feature 1..20: note = phosphorothioate bonds between bases
  source 1..20: mol_type = other DNA
  modified_base 1: mod_base = OTHER; note = 2'-O-methyl guanosine
  modified_base 2: mod_base = OTHER; note = 2'-O-methyl guanosine
  modified_base 3: mod_base = OTHER; note = 2'-O-methyl adenosine
  modified_base 4: mod_base = OTHER; note = 2'-O-methyl adenosine
  modified_base 5: mod_base = OTHER; note = 2'-O-methyl uracil
  modified_base 16: mod_base = OTHER; note = 2'-O-methyl uracil
  modified_base 17: mod_base = OTHER; note = 2'-O-methyl adenosine
  modified_base 18: mod_base = OTHER; note = 2'-O-methyl adenosine
  modified_base 19: mod_base = OTHER; note = 2'-O-methyl uracil
  modified_base 20: mod_base = OTHER; note = 2'-O-methyl guanosine
ggaanggttacaaaanaang

SEQ ID NO: 8 - DNA, 20 bp, synthetic construct
  misc_feature 1..20: note = antisense oligonucleotide gapmer 6
  misc_feature 1..20: note = phosphorothioate bonds between bases
  source 1..20: mol_type = other DNA
  modified_base 1: mod_base = OTHER; note = 2'-O-methyl guanosine
  modified_base 2: mod_base = OTHER; note = 2'-O-methyl uracil
  modified_base 3: mod_base = OTHER; note = 2'-O-methyl adenosine
  modified_base 4: mod_base = OTHER; note = 2'-O-methyl adenosine
  modified_base 5: mod_base = OTHER; note = 2'-O-methyl guanosine
  modified_base 16: mod_base = OTHER; note = 2'-O-methyl cytosine
  modified_base 17: mod_base = OTHER; note = 2'-O-methyl cytosine
  modified_base 18: mod_base = OTHER; note = 2'-O-methyl adenosine
  modified_base 19: mod_base = OTHER; note = 2'-O-methyl adenosine
  modified_base 20: mod_base = OTHER; note = 2'-O-methyl guanosine
gnaagattttccacaacaag

SEQ ID NO: 9 - DNA, 20 bp, synthetic construct
  misc_feature 1..20: note = Antisense oligonucleotide gapmer 7
  misc_feature 1..20: note = phosphorothioate bonds between bases
  source 1..20: mol_type = other DNA
  modified_base 1: mod_base = OTHER; note = 2'-O-methyl uracil
  modified_base 2: mod_base = OTHER; note = 2'-O-methyl adenosine
  modified_base 3: mod_base = OTHER; note = 2'-O-methyl adenosine
  modified_base 4: mod_base = OTHER; note = 2'-O-methyl guanosine
  modified_base 5: mod_base = OTHER; note = 2'-O-methyl adenosine
  modified_base 16: mod_base = OTHER; note = 2'-O-methyl cytosine
  modified_base 17: mod_base = OTHER; note = 2'-O-methyl cytosine
  modified_base 18: mod_base = OTHER; note = 2'-O-methyl adenosine
  modified_base 19: mod_base = OTHER; note = 2'-O-methyl uracil
  modified_base 20: mod_base = OTHER; note = 2'-O-methyl uracil
naagaagtagatggcccann

SEQ ID NO: 10 - DNA, 106 bp, synthetic construct
  source 1..106: mol_type = other DNA
  misc_feature 1..106
ttattgaactcccattccgtatatgacattatagtcagcattcttggggaaacagagatgactaagtaagattattgccttctaggaggttatagtctagcaggag

SEQ ID NO: 11 - DNA, 121 bp, synthetic construct
  source 1..121: mol_type = other DNA
gagaccgggacttgctgtgttgcccaggctggtcttgaactcctggtcttaagcaatcatactgctttggccacccaaagcactgggattacaggcgtgaaccaccacacccagctcttaa

SEQ ID NO: 12 - DNA, 23 bp, synthetic construct
  misc_feature 1..23: note = ALOX12-AS1-201 forward primer
  source 1..23: mol_type = other DNA
gggctcttagtatcggaggattg

SEQ ID NO: 13 - DNA, 22 bp, synthetic construct
  misc_feature 1..22: note = ALOX12-AS1 201 reverse primer
  source 1..22: mol_type = other DNA
gcccaacagtaaggtggataga

SEQ ID NO: 14 - DNA, 23 bp, synthetic construct
  misc_feature 1..23: note = ALOX12-AS1-202 forward primer
  source 1..23: mol_type = other DNA
gctcttagtatcggaggattgga

SEQ ID NO: 15 - DNA, 24 bp, synthetic construct
  misc_feature 1..24: note = ALOX12-AS1-202 reverse primer
  source 1..24: mol_type = other DNA
cagagctcagaaaggagatgaaac

SEQ ID NO: 16 - DNA, 21 bp, synthetic construct
  misc_feature 1..21: note = ALOX12-AS1-203 forward primer
  source 1..21: mol_type = other DNA
aaagtctaactggcggaactc

SEQ ID NO: 17 - DNA, 26 bp, synthetic construct
  misc_feature 1..26: note = ALOX12-AS1-203 reverse primer
  source 1..26: mol_type = other DNA
cttatgctaaggtttcaggtgtattg

SEQ ID NO: 18 - DNA, 24 bp, synthetic construct
  misc_feature 1..24: note = ALOX12-AS1-204 forward primer
  source 1..24: mol_type = other DNA
acctgaaattccttagtctctcct

SEQ ID NO: 19 - DNA, 20 bp, synthetic construct
  misc_feature 1..20: note = ALOX12-AS1-204 reverse primer
  source 1..20: mol_type = other DNA
agttcctcaatggtgccaac

SEQ ID NO: 20 - DNA, 21 bp, synthetic construct
  misc_feature 1..21: note = ALOX12-AS1-205 forward primer
  source 1..21: mol_type = other DNA
aaagtctaactggcggaactc

SEQ ID NO: 21 - DNA, 26 bp, synthetic construct
  misc_feature 1..26: note = ALOX12-AS1-205 reverse primer
  source 1..26: mol_type = other DNA
cttatgctaaggtttcaggtgtattg

SEQ ID NO: 22 - DNA, 23 bp, synthetic construct
  misc_feature 1..23: note = ALOX12-AS1-206
  source 1..23: mol_type = other DNA
gggctcttagtatcggaggattg

SEQ ID NO: 23 - DNA, 22 bp, synthetic construct
  misc_feature 1..22: note = ALOX12-AS1 206 reverse primer
  source 1..22: mol_type = other DNA
tgaagggaggaaatggtagctg

SEQ ID NO: 24 - DNA, 22 bp, synthetic construct
  misc_feature 1..22: note = ALOX12-AS1-207 forward primer
  source 1..22: mol_type = other DNA
tccttagtctctcctgtgttgg

SEQ ID NO: 25 - DNA, 23 bp, synthetic construct
  misc_feature 1..23: note = ALOX12-AS1-207 reverse primer
  source 1..23: mol_type = other DNA
gttcagctaccagttcctcaatg

SEQ ID NO: 26 - DNA, 24 bp, synthetic construct
  misc_feature 1..24: note = ALOX12-AS1-208 forward primer
  source 1..24: mol_type = other DNA
acctgaaattccttagtctctcct

SEQ ID NO: 27 - DNA, 24 bp, synthetic construct
  misc_feature 1..24: note = ALOX12-AS1-208 reverse primer
  source 1..24: mol_type = other DNA
actgttctctccttagtagtgtgg

SEQ ID NO: 28 - DNA, 23 bp, synthetic construct
  misc_feature 1..23: note = ALOX12-AS1-209 forward primer
  source 1..23: mol_type = other DNA
gctcttagtatcggaggattgga

SEQ ID NO: 29 - DNA, 25 bp, synthetic construct
  misc_feature 1..25: note = ALOX12-AS1-209 reverse primer
  source 1..25: mol_type = other DNA
gtgtattgatggtatccagatcagc

SEQ ID NO: 30 - DNA, 22 bp, synthetic construct
  misc_feature 1..22: note = GAPDH forward primer
  source 1..22: mol_type = other DNA
acgggaagcttgtcatcaatgg

SEQ ID NO: 31 - DNA, 22 bp, synthetic construct
  misc_feature 1..22: note = GAPDH reverse primer
  source 1..22: mol_type = other DNA
ccagtagaggcagggatgatgt

SEQ ID NO: 32 - DNA, 24 bp, synthetic construct
  misc_feature 1..24: note = ALOXE3 forward primer
  source 1..24: mol_type = other DNA
gacgtcaacagctttcaggagatg

SEQ ID NO: 33 - DNA, 23 bp, synthetic construct
  misc_feature 1..23: note = ALOXE3 reverse primer
  source 1..23: mol_type = other DNA
agatcgtcttggtggctgagtat

SEQ ID NO: 34 - DNA, 22 bp, synthetic construct
  misc_feature 1..22: note = ALOX5 forward primer
  source 1..22: mol_type = other DNA
ggcaggaagacctgatgtttgg

SEQ ID NO: 35 - DNA, 22 bp, synthetic construct
  misc_feature 1..22: note = ALOX5 reverse primer
  source 1..22: mol_type = other DNA
gcagctcaaagtccacgatgaa

SEQ ID NO: 36 - DNA, 24 bp, synthetic construct
  misc_feature 1..24: note = ALOX12 forward primer
  source 1..24: mol_type = other DNA
gagaagaggctggactttgaatgg

SEQ ID NO: 37 - DNA, 24 bp, synthetic construct
  misc_feature 1..24: note = ALOX12 reverse primer
  source 1..24: mol_type = other DNA
ccattgaggaactggtagctgaac

SEQ ID NO: 38 - DNA, 20 bp, synthetic construct
  misc_feature 1..20: note = RNASEK forward primer
  source 1..20: mol_type = other DNA
gctttctcccaccgctttcg

SEQ ID NO: 39 - DNA, 22 bp, synthetic construct
  misc_feature 1..22: note = RNASEK reverse primer
  source 1..22: mol_type = other DNA
ccgaacttggcagaaagagaag

SEQ ID NO: 40 - DNA, 15 bp, synthetic construct
  misc_feature 1..15: note = ALOX12 F1 sequencing primer
  source 1..15: mol_type = other DNA
taccgcatccgcgtg

SEQ ID NO: 41 - DNA, 15 bp, synthetic construct
  misc_feature 1..15: note = ALOX12F2 sequencing primer
  source 1..15: mol_type = other DNA
tacaaccgcgtgcag

SEQ ID NO: 42 - DNA, 18 bp, synthetic construct
  misc_feature 1..18: note = ALOX12F3 sequencing primer
  source 1..18: mol_type = other DNA
caggatgatgagttgttc

SEQ ID NO: 43 - DNA, 18 bp, synthetic construct
  misc_feature 1..18: note = ALOX12F4 sequencing primer
  source 1..18: mol_type = other DNA
ctcgttatgctgaagatg

SEQ ID NO: 44 - DNA, 16 bp, synthetic construct
  misc_feature 1..16: note = ALOX12F5 sequencing primer
  source 1..16: mol_type = other DNA
tcttctccgggtcgta

SEQ ID NO: 45 - DNA, 17 bp, synthetic construct
  misc_feature 1..17: note = ALOX12R1 sequencing primer
  source 1..17: mol_type = other DNA
ctgttctctatgcagct

SEQ ID NO: 46 - DNA, 18 bp, synthetic construct
  misc_feature 1..18: note = ALOX12R2 sequencing primer
  source 1..18: mol_type = other DNA
ctgaagttctttctccag

SEQ ID NO: 47 - DNA, 18 bp, synthetic construct
  misc_feature 1..18: note = ALOX12R3 sequencing primer
  source 1..18: mol_type = other DNA
catcttcagcataacgag

SEQ ID NO: 48 - DNA, 450 bp, synthetic construct
  misc_feature 1..450: note = ALOX12-AS1-201 RNA FISH probe sequence
  source 1..450: mol_type = other DNA
tgaactcccattccgtatatgacattatagtcagcattcttggggaaacagagatgactaagtaagattattgccttctaggaggttatagtctagcaggaggactgtgaaccctcccagcctgccgctcgctagctggagtgccctgatgtcaggtggaaggcaggagggactggaagaagggggtccagaaggaggccctaacagtggtgtgggagaaacctgctgctttggggtcggcgctcagatggtggtggaatgtgaaggagaagtaggaggtgtaagattattgacagttcttgtcacctgactgttaagaatagtgagagaaggccaggtgcagtggctcacacctgtaatcccagcactttgggaggccgaggcaggcagattacttgagcccagaagttcaagaccagcctgggcaatatggcgaaaccccatctctac

SEQ ID NO: 49 - DNA, 432 bp, synthetic construct
  misc_feature 1..432: note = ALOX12-AS1-202 RNA FISH probe sequence
  source 1..432: mol_type = other DNA
agacagcatctcgtatgttgcccaggctggccttgaaagcctggcctcaagccatcttcctgcctcagcctcccaagtagctgggatcacagggttgtggcatcacagctggctatattcttaacattattttgtaaccattccaacccccagaaatttctctctggctgacttgatccacagcgcctccatcgccatccctgagtgccttgttgtggaaaatcttactttatcttggttctgtttggtataatcggggaaagtctgtattctttcattatgtaaaacaacttatctctcattgtttcatctcctttctgagctctgctctgccagctctctttccaaaaccaaaatggctcttcaagttattttgtaaataataatgggccatctacttcttaacataaatgaatgattttccaaggtcaa